(19) United States
(12) Patent Application Publication
(10) Pub. No.: US 2026/0165842 A1
(43) Pub. Date: Jun. 18, 2026

(54) MULTI-MATERIAL STIFFNESS-MATCHED IMPLANT DEVICES AND FABRICATION METHODS

(71) Applicant: OHIO STATE INNOVATION FOUNDATION, Columbus, OH (US)

(72) Inventors: David DEAN, Upper Arlington, OH (US); Agnieszka CHMIELEWSKA-WYSOCKA, Columbus, OH (US); Alan LUO, Westerville, OH (US); Boyd PANTON, Dublin, OH (US); Luis Hector OLIVAS, Columbus, OH (US); Ciro RODRIGUEZ, Columbus, OH (US); Antonio RAMIREZ, Dublin, OH (US)

(21) Appl. No.: 19/129,417

(22) PCT Filed: Nov. 13, 2023
(86) PCT No.: PCT/US2023/079498

§ 371 Date: May 13, 2025

Related U.S. Application Data
(60) Provisional application No. 63/383,517, filed on Nov. 14, 2022.

(51) Int. Cl. A61F 2/28; A61F 2/30; A61L 27/04; A61L 27/06; A61L 27/10; A61L 27/12; A61L 27/28; A61L 27/54; A61L 27/58; B32B 15/01

(52) U.S. Cl. CPC A61F 2/2846; A61L 27/047; A61L 27/06; A61L 27/10; A61L 27/12; A61L 27/28; A61L 27/54; A61L 27/58; B32B 15/01; A61F 2002/2835; A61F 2002/285; A61F 2002/30032; A61F 2002/30331; A61F 2002/30784; A61F 2002/3092; A61F 2002/30971; A61L 2400/16; B32B 2250/02; B32B 2255/06; B32B 2305/026; B32B 2307/7163; B32B 2311/005; B32B 2311/20; B32B 2311/22; B32B 2535/00

(57) ABSTRACT

A device for bone graft fixation, replacement, or abutment, includes a non-resorbable portion made of a non-resorbable biocompatible material and a resorbable portion made of a resorbable biocompatible material joined to the non-resorbable portion. The resorbable portion is resorbed in a pre-determined timing such that mechanical properties of the device are modulated to avoid stress shielding of a bone and to control stress concentrations in the device below a predetermined level to avoid failure of the device.

### PRIOR RELATED APPLICATIONS

The present application claims the priority to U.S. Provisional Application No. 63/383,517, filed on Nov. 14, 2022, entitled Multi-Material Stiffness-Matched Implant Devices and Fabrication Methods, which is incorporated herein in its entirety.

### FIELD OF INVENTION

The present disclosure generally relates to devices for skeletal reconstruction and methods of making those devices. More specifically, the present disclosure relates to multi-material stiffness-matched devices for skeletal reconstruction surgery that change mechanical properties during the healing process or, as in many cases is preferable, after the healing process is complete. The stiffness-matched devices are configured to ensure healing and restoring normal function of the healing skeleton during and after the period of healing.

### BACKGROUND

To repair fractured diseased bones, following bone tumor resection, or during joint replacement surgery, implants such as bone plates and screws are used to stabilize a site where the skeleton is repaired and/or reconstructed, help maintaining contact between bone fragments, and allow post-operative weight bearing and patient mobility. In an ideal scenario, following the healing period, the implants should allow the loading pattern of the healed bone to return to normal (i.e., to transfer as much of the portion of the load that the fixation had carried during healing to the healed bone) rather than causing bone stress shielding of that bone or stress concentration in the implant, as either may lead to failure of the bone and/or of the implant. It would be beneficial to have implants that are designed and fabricated to accommodate the patient's changing functional needs during bone healing and post-healing.

### SUMMARY

In one embodiment, a device for bone graft fixation, replacement, or abutment, includes a non-resorbable portion made of a non-resorbable biocompatible material and a resorbable portion made of a resorbable biocompatible material joined to the non-resorbable portion. The resorbable portion is resorbed in a pre-determined timing such that mechanical properties of the device are modulated to avoid stress shielding of a bone and to control stress concentrations in the device below a predetermined level to avoid failure of the device.

In another embodiment, a method of fabricating a device for bone graft, replacement, or abutment, includes obtaining a non-resorbable portion made of a non-resorbable biocompatible material. The method includes obtaining a resorbable portion made of a resorbable biocompatible material. The resorbable portion is configured to be resorbed in a pre-determined timing such that mechanical properties of the device are modulated to avoid stress shielding of a bone and to control stress concentrations in the device below a predetermined level to avoid failure of the device. The method also includes joining the non-resorbable portion and the resorbable portion to form the device.

### BRIEF DESCRIPTION OF THE DRAWINGS

FIG. 1 is a perspective view of an example of a stiffness-matched device.

FIG. 2 is a perspective view of another example of a stiffness-matched device, in which the resorbable material is housed within the device rather than sitting outside of it (as in FIG. 1).

FIG. 3 is a perspective view of another example of a stiffness-matched device, in which the resorbable material is housed by slats to prevent dislodging of large pieces of resorbing materials.

FIG. 4A and FIG. 4B are, respectively, exploded perspective view and side view of another example of a stiffness-matched device having a solid non-resorbable portion and a resorbable portion having a scaffold structure or a structure with planned porosity.

FIG. 5A is an exploded perspective view of another example of a stiffness-matched device that may be used as a proximal hip replacement device that translates directly to a cortical bone.

FIG. 5B is a perspective view of the stiffness-matched device of FIG. 5A in an example application.

FIG. 5C is a perspective view of another example of a stiffness-matched device.

FIG. 6 is a flow chart of an example of fabricating and using a stiffness-matched device.

FIG. 7 shows an example of a stiffness-matched device manufactured via additive manufacturing and press fitting techniques.

FIG. 8 shows another example of a stiffness-matched device manufactured via additive manufacturing and press fitting techniques.

FIG. 9 shows an example of virtual surgical planning of a stiffness-matched device in a rat model sample.

FIG. 10 shows example NiTi gyroid structures of different porosities manufactured by laser powder bed fusion (LPBF) process.

FIG. 11 shows an example of varying mechanical properties of a gyroid structure by changing the porosity.

FIG. 12 shows mechanical testing results of example gyroid structures under four-point bending.

FIG. 13 shows an example manufacturing method for forming an interlayer between non-resorbable and resorbable portions of a stiffness-matched device.

FIG. 14 shows an example virtual surgical planning process for implanting a stiffness-matched device disclosed herein.

FIG. 15 shows an example implant design process of a stiffness-matched bone fixation for mandibular bone.

FIG. 16 shows personalized implant designs at different implant locations.

FIG. 17 shows virtual surgical planning of a mandibular model.

FIG. 18 shows modeled Von Mises stress distribution of the mandibular model of FIG. 17 before bone healing for different implant locations and material choices.

FIG. 19 shows example modeled Von Mises stress distribution of the mandibular model of FIG. 17 post bone healing for different implant locations and material choices.

FIG. 20A shows Von Mises analysis (maximum equivalent Von Mises stress) of the mandibular model of FIG. 17 before bone healing for different implant locations and material choices.

FIG. 20B shows maximum anterior and posterior gap of the mandibular model of FIG. 17 during unilateral clenching for different implant locations and material choices.

FIG. 21 shows resulting stiffness-matched device deformation values of the mandibular model of FIG. 17 post bone healing for different implant locations and material choices.

### DETAILED DESCRIPTION

The present disclosure is not limited in terms of the particular embodiments described in this application, which are intended as illustrations of various aspects only. Many modifications and variations can be made without departing from the scope of the invention, as will be apparent to those skilled in the art. Functionally equivalent methods within the scope of the disclosure, in addition to those enumerated herein, will be apparent to those skilled in the art from the following descriptions. Such modifications and variations are intended to fall within the scope of the appended claims. The terminology used herein is for the purpose of describing particular embodiments only and is not intended to be limiting.

The present disclosure generally relates to multi-material stiffness-matched devices for fixation or other skeletal reconstruction procedures that have a specific role to play during and after skeletal reconstruction surgery through the time point when bone graft healing or bone replacement device integration is complete. It is anticipated that there will be a change, a reduction of mechanical properties, once the healing process has progressed to the point that the fixated bones are sufficiently healed to undergo their normal, i.e., non-fixated, loading pattern. The main purpose of a skeletal reconstruction device is to ensure complete healing of fractured, engrafted, or otherwise, surgically approximated bone and/or skeletal reconstruction devices. In this broader application of skeletal reconstruction, in addition to biocompatibility, equal significance should be given to whether the device is intended to provide immobilization or replacement of skeletal function. Both types of devices require immobilization through the healing process. Thereafter the role played by the resorbable component will be different. Repair of fractures or engraftment would play little to no role due to the loss of the resorbable component. Whereas devices such as partial or total joint replacement, stabilization device, defect insert-implant, percutaneous device mooring sites would become better integrated and able to bear the subsequent, healed, load following the healing process.

Adequate stabilization preserves the optimal alignment and apposition of healing bone while it continues to experience tolerable levels of physiologic loading. A stiffness-matching approach to fixation assumes that the insurance of approximation of bone segments intended to heal together, or a skeletal reconstruction device that must integrate with host bone, is not inconsistent with a future result where the bone-bone or bone-device junction has healed allowing a return to the normal loading pattern (i.e., no longer requiring fixation of osteotomies, or open host bone-reconstructive device connections, and/or fracture sites which are no longer open, devoid of bone or are no longer present). One way to conceive of this is to consider an offloading of the fixation or attachment site or free bone graft (i.e., bone fragments) (i.e., fragments placed in fixation to facilitate graft-host, graft-graft, or device-host healing together) to the normal stress-strain trajectory of fully-loaded, fully-utilized, reconstructed, and healed bone or bone plus device reconstructed anatomy. However, initially, stability at the device/bone or bone/bone junction is essential for the formation of bridging bone (i.e., bridging the device bone junction, a fixated osteotomy site, or a fixated fracture site, thereby bringing about bone union in the healing site) and possibly a somewhat larger callus that assists in bridging the fracture or device-bone site. Stability can be achieved by directing as much load as possible towards ensuring compression, not tension, at the healing site. Once healed, the previous preferred loading pattern (compression at the site of healing) and subsequently the load can be taken-up by the newly healed bone segment in the normal stress-strain pattern, thereby “off-loading” the fixation device. In the case of restorative implants (e.g., hip, knee, spine, shoulder), a sustainable loading of the implant is sought. As the bone and any additional callus remodels, stronger cortical bone will form where needed, allowing greater and greater loads to be transmitted across the previous fracture or engraftment boundaries (i.e., through the newly healed site), and the restoration of normal loading geometry and muscle recruitment, for ambulatory or other function. This accomplished/newly healed bone tissue can begin to undergo continuous, healthy remodelling in response to restoration of normal stress-strain trajectories. Once the bone is under a normal loading regime, muscle power can be restored, perhaps very close to or reaching prior power. This type of remodelling, healthy bone turnover due to regular use, minimizes re-fracture or device failure following bone healing.

Although standard-of-care skeletal reconstruction devices, sometimes with additional stabilization (e.g., casts or mandibulo-maxillary fixation), will usually allow normal healing and improved comfort for the patient during normal activities, there may be significant disadvantages during long-term use. For example, abnormal loading created by overly stiff skeletal devices may prolong the loading regime seen during the healing process and lead to the failure of the fixation device.

Stress concentration in the device, especially in small regions that cannot tolerate a high load, may lead to failure or potentially rapid failure of the device. Devices that carry high loads for too long (i.e., fail to transfer the load back to the healed bone) may fail due to fatigue. Meanwhile, stress concentration in the surrounding bone, for example at the sharp edge of screw threads, can lead to loss of contact and disassociation of the device from the bone during the healing period. Thus, often there is an emphasis on “bicortical” screw placement. However, our approach of stiffness-matching the implant better allows the whole implant to respond to constantly changing load conditions than a highly stiff and strong device that tends to concentrate load to where it is first received (i.e., stress concentration). In addition to bicortical screw placement, sufficiently flexible regions between screws, rigid areas around them, and non-aligned screw placement, such as in many wrap-around devices such as for ribs, can help ensuring that fewer screws fail. However, it may be useful to also have the plate fail at a later point in response to fully functional (healed bone) and its priority to handle normal loading. Thus, strategic weakening, post-healing, of fixation devices may be a useful strategy in bones that, when healthy, are under significant (i.e., major load bearing segment of the skeleton) loads.

Stress shielding of bone can manifest as the reduction of bone density, and eventually resorption seen as loss of bone volume, due to the redistribution of load that occurs when a skeletal device takes over the load that is normally received by bone. According to Wolff's law, bones undergo regeneration and remodelling, and adapt their architecture, mass, and mechanical properties in response to mechanical load. A skeletal device constructed of a material that is much stiffer than surrounding bone, may cause a non-physiological (i.e., abnormal) redistribution of force transmission and prevents the local bone from receiving sufficient mechanical stimulus during the remodelling phase of healing and thereafter. As a result, the healed segment, and possibly adjacent segments, of bone may resorb over time. A decrease in bone mass and density may also contribute to bone implant failure (e.g., screw loosening and/or plate cracking) prior to complete bone healing. The resorptive process generated by stress shielding may contribute to problems that lead to bone loss and revision surgery. This is commonly seen in the following skeletal regions: craniomaxillofacial, knee, hip, shoulder, forearm/wrist, and spine. Therefore, mechanical properties and geometry are critical factors that are considered during design of the stiffness-matched devices disclosed herein to avoid stress shielding of bone and stress concentration in and adjacent skeletal reconstruction (immobilization) or replacement devices.

The mechanical performance of a skeletal fixation or a skeletal replacement device (e.g., a total or partial joint replacement device) can be modified by the device's location, geometry, or the material employed. The Young's modulus of metallic materials in routine use for skeletal fixation or reparative devices is often dramatically higher than the modulus of bone. Titanium alloys such as commonly used Ti-6A1-4V, cobalt-chromium alloys, and stainless steel have Young's moduli of about 110, 190, and 210 GPa, respectively, whereas that of most cortical bone are in the 20-25 GPa range and possibly 15-30 GPa range. Therefore, it is beneficial to develop materials that have an elastic modulus that will avoid stress shielding of the bone and avoid stress concentration in the device (e.g., control stress concentration in the device below a certain critical level to avoid pre-matured failure of the device) and promote speed healing.

The concept of “stiffness matching” as disclosed herein and the design of a “stiffness matched” device to be implanted during surgery is not merely to have fixation hardware in the range of bone, but rather to match what is needed to heal the bone following skeletal reconstructive surgery. In addition to maintaining fractures or grafted bone in compression (i.e., not under tension) and the restoration of stress-strain trajectories in the healing bone can also be facilitated by modifying the geometry of an immobilization and/or replacement device. One such strategy is the introduction of carefully designed pore geometry, large hollow regions, or lattice structures that will allow obtaining predefined mechanical properties that best lead to rapid healing and avoidance of bone stress shielding or device stress concentrations. The combination of low modulus metallic materials and personalized design of device geometry are a good start to the design of stiffness-matched medical devices that would both facilitate bone healing and recreate the desirable stress-strain trajectory experienced by normal bone.

The “stiffness matching” approach disclosed herein is not merely a matching of stiffness of the device to the adjacent bone. The optimal mechanical properties may vary depending on the stages of healing (pre-healing and post-healing). In regard to pre-healing, the goal of stiffness matching is to bring about compression of the fracture site and/or host and bone graft, without allowing for opening of the bone wound site, which may occur under tension. The optimal geometry and material properties, including whether it will be useful to have a resorbable component, will likely differ depending on the (1) location, (2) geometry, and (3) material used in the device. If the goal of the device is fixation of a fracture or a graft, it may be best if it becomes mechanically irrelevant, or at least inconsequential, once the bone is fully healed. Whereas, if the device is intended to help a skeletal replacement device better integrate a non-resorbing component integrate into the skeleton, it may be useful to have the remaining portion (e.g., skeletal fixation for an under supportive portion of the skeleton, joint replacement, osseointegrated device for percutaneous fixation or prosthesis, etc.) to be well integrated (e.g., via resorbable surfaces such as coatings) or interdigitating slivers that draw bone into the device, being replaced by bone to enhance integration. The remaining device, especially in the post-healing phase, and possibly during healing, may be stiff and strong in its post-healing performance.

The present disclosure offers a novel method for a stiffness-matched approach, a new solution to mitigate or eliminate negative effects currently associated with long-term use of skeletal reconstructive devices. A stiffness-matched device disclosed herein is a strength modulated device that is configured to restore natural force transmission through healed bone.

It is also an unprecedented approach in the use of resorbable magnesium alloys to consider the combination of a resorbable component much like a bone graft. That is, on initial placement, it comes under a normal load, but after healing the healed region is expected undertake most if not all loading. To date resorbable metal devices, primarily Mg and Zn alloys, have primarily been single material devices. Moreover, the clinically most successful alloys often include rare earth materials. Thus, the resorbable components tend to be small in size, perhaps to limit the amount of toxic material that is absorbed by the patient's tissues. In the present disclosure, since the alloy utilized in the applications, has only nutritive elements that are safe when absorbed in relatively small amounts per day locally, it is possible to safely increase the resorbable component's size. This allows use of a resorbable component at a large size relative to that made of rare earth materials, or other toxic-bearing materials. Thus, while the device in the present disclosure is able to resist placing the healing site under tension and translate as much load as possible to keeping the healing site in compression, the subsequent resorption of the resorbable component will additionally ensure that the healed bone will not be stress shielded.

From the clinical point of view, there is a strong need for skeletal reconstructive devices that minimize fracture or bone-implant interface micromotion throughout the bone healing process, while adjusting its mechanical properties to the healed bone to restore proper stress-strain trajectories (post-healing) and transfer force transmission over time from the restorative device to the healed bone. One way to do that is to have a resorbable portion of the fixation device resorb after the bone has healed. In the present disclosure, a stiffness-matched device disclosed herein may be fabricated from material that may change its properties over time, including partially resorbable, multi-material (metal-metal, metal-polymer, metal-ceramic, metal composite, etc.) that is stiffness-matched to achieve the desired function of the healed bone, where the remaining portion of the device does not interrupt the normal stress-strain trajectory of the healed bone. If it is a skeletal replacement rather than skeletal restorative/reconstructive device, the device disclosed herein may also include non-resorbable component(s) and with the incorporation of the non-resorbable component(s), the function of the device is improved following bone healing.

The stiffness-matched device disclosed herein is a strength and stiffness-modulated medical device for skeletal reconstruction surgery that changes its mechanical properties during the healing process. The device initially performs the work of keeping at least one fracture site (full or partial), a segmental defect, a percutaneous implant, two or more healing sites of a bone graft, or a total joint replacement and adjacent bone in close opposition. This helps bone healing or “union” of bone segments in medical parlance, following reconstructive surgery. Following the healing period, the device is designed to allow the loading pattern of the healed bone to return to normal (i.e., to transfer as much as possible of the portion of the load that the fixation had carried during healing to the healed bone) rather than causing bone stress shielding or stress concentration in the device leading to rapid failure or fatigue failure of the device. One of the key steps in designing the stiffness-matched device disclosed herein involves joining a resorbable component that once resorbed is intended to ensure that the device no longer carries a significant load (i.e., stiffness and strength need to ensure immobilization) nor interrupts the normal stress-strain trajectories of the healed bone.

The stiffness-matched device disclosed herein is configured to eliminate or mitigate the stress shielding phenomena (i.e., bone resorption). The stiffness-matched device disclosed herein is also configured to eliminate or mitigate the stress concentration phenomena that may lead to the device failure. Furthermore, the stiffness-matched device disclosed herein is configured to provide mechanical and shape personalization produced in sufficient time (e.g., point-of-care manufacturing) for patients (e.g., cancer, trauma) for whom personalized devices are otherwise unavailable due to a lengthy fabrication process.

Personalization of a fixation device will help implement the intent of a previously conceived, virtual surgical plan (e.g., computer-planning of a surgical procedure based on a pre-operative image such as a 3D CT image). A pre-operatively prepared device allows shortening the length of a skeletal reconstruction procedure, thereby decreasing the risk to patients, as opposed to the surgeon manually fashioning (i.e., hand-bending) the fit of an implantable device or the skeleton to receive the device. Alternatively, off-the-shelf devices could be modified to include resorbable components and still benefit from the loss of the resorbable component of the device disclosed herein.

In one embodiment, the stiffness-matched device disclosed herein is modulated both in strength and stiffness and is made of inert biocompatible superstructure made of polymer, metal/alloy (e.g., titanium and its alloys such as Ti-6A1-4V, Ni—Ti, Ti—Nb, cobalt-chromium alloys, stainless steel, etc.) and/or a composite material that is initially reinforced by a bioresorbable metal attachment or insert. The insert initially adds stiffness and strength to the device in the same or a similar way to how a bone graft brings strength to a segmental defect. Once the resorbable component goes away, the device is incapable of bearing its original load. It could then allow the healed bone to take over its original function.

Historically, the terms resorption (i.e., process of losing substance), adsorption (i.e., adhesion in an extremely thin layer of molecules to the surfaces of solid bodies-the mechanism of cell absorption), and absorption (i.e., actual intake, the process of passing through a structure such as the intestinal or a cell wall) have been used interchangeably in reference to degradable polymers. None speaks to the process of degradation or incorporation. Hydrolysis (i.e., chemical process of decomposition involving the splitting of a bond due to the invasion of water and possibly swelling and the incorporation water in the degradation byproducts) and erosion (i.e., breaking apart of a surface, perhaps in response to enzymatic or mechanical activity) is the process by which most currently used degradable polymers or body structures themselves (e.g., bone resorption) degrade. The use of resorbable metals has been long investigated, especially for skeletal reconstruction or replacement (e.g., approximately 20 years). However, medical users of these materials are not as aware of the nature of the various processes of metallic corrosion such as galvanic (i.e., spontaneous redox reaction), electrolytic (due to the presence of an electric current), tribocorrosion (rubbing wear), or rusting (i.e., deterioration due to oxidation). Indeed, at its most general level, corrosion most often is used to refer to an active eating away of structure. More strictly, it is a process in which metals in manufactured states return to their natural oxidation states. Careful documentation of the process and speed of degradation of weight and strength of polymer, metal, or ceramic resorbable devices and whether the byproducts of degradation are incorporated by tissues in the body or removed (e.g., metabolism, excretion, exhalation, defecation, etc.) is required.

Similarly, in the example of percutaneous implants, an “offloading” of load-bearing would occur in a fluted cuff device with resorbable and inert fingers in the cortex, where the resorbable portion encourages bone ingrowth as it is resorbed. These fingers may be threaded (e.g., cylindrical or semicylindrical screw or screw-like), textured, or made porous to provide better attachment and load transfer. The resorbable portion may be designed with texture and/or porosity to assist the resorption. Metallic, inert struts could continue to direct and carry load, perhaps in preferred directions to assist in load transfer between the bone and the implant, providing long-term stability. Thus, directional and designed porosity (e.g., uniform or directional gradient gyroid, adaptive gyroid, or other load directing porous structures) could be filled with a resorbable component (e.g., metallic, ceramic, resorbable polymer, and/or with bioactive components such as ligands, growth factors, exosomes, etc.) that could be used to enhance bone ingrowth and strengthening to ensure the success of the interface of the percutaneous device and the boney component. Ingrowth of strong bone where needed could help stabilize the connection of the skeleton with a percutaneous device to provide external fixation, for example, for a prosthetic limb, or traction (e.g., Ilizarov distraction technique), or another type of device. The overall percutaneous, bone-moored device is not intended to mimic the natural human bone, but rather to perform a function, i.e., healing during fracture and/or segmental defect repair, or integration of an abutment for an external removable or permanent device (e.g., a prosthetic limb). The function, loading, and muscle power that can act on, of such a device is constantly adjusting and changing as bone heals and strengthens through bone remodeling in response to its use. For the stiffness-matched devices disclosed herein, the changing mechanical performance of the device during the healing process are facilitated by the use of strategic placement of relatively low (i.e., similar to bone) modulus materials and predefined geometries. This is different from conventional devices which tend to be mounted in the medullary (marrow) cavity in ways similar to proximal femur hip implants.

In one embodiment, the multi-material fixation, abutment, or replacement device is fabricated, and the two components (superstructure and resorbable component) are joined by any suitable method or methods, such as press fitting, casting, infiltration, welding, brazing, explosive joining, impact welding including: vaporizing foil actuator welding, magnetic impulse welding, and laser driven impact welding, pulsed and continuous wave laser welding, electron beam welding, friction welding, ultrasonic welding, ultrasonic additive manufacturing, cold spray, infusion, jetting, soldering, diffusion joining, transient liquid bonding or a variety of additive manufacturing technologies. A resorbable “insert” region may be caged by the inert material to ensure that the resorbable insert region does not fracture or otherwise degrade into smaller pieces that can then break away and lodge in nearby tissues. Rather, it should degrade, predictably, in place with no toxic byproducts or dangerous degradation or wear detritus. The alloys used to construct the device disclosed herein should not produce toxic use or degradation byproducts, or byproducts that cannot be metabolized safely at the rate at which they are released into the adjacent tissue and/or be safely excreted if they enter the interstitial or vascular space. Conventionally, as an example, the joining of nickel titanium (NiTi) 3D printed structures with pure magnesium (Mg) is unlikely a suitable material candidate. However, a high strength joint, with damping capacity, and energy absorption efficiency applications may be obtained by overmolding. Thus, the joining of NiTi 3D printed structures with pure Mg or a biocompatible Mg alloy may be overmolded. While pure Zn is equally unlikely a suitable material candidate, a Zn-based alloy with or without Mg, like pure Mg or a biocompatible and resorbable Mg alloy, could also be dedicated for biomedical fixation, replacement, or abutment applications. The formation of brittle phases during joining should be avoided in these applications. The stiffness-matched device disclosed herein is configured to exclude, prevent, or be substantially free of intermediate phase formation. Herein “substantially free of intermediate phase formation” means even if the intermediate phase is formed during the joining process, the relative portion of the intermediate phase is sufficiently low such that its presence does not negatively impact the functionality/modulated mechanical properties of the device. “Substantially free” of intermediate phase formation herein may refer to intermixing width or interface width between the resorbable and non-resorbable materials between 0 μm and 80 μm, between 0 μm and 70 μm, between 0 μm and 60 μm, between 0 μm and 50 μm, between 0 μm and 40 μm, between 0 μm and 30 μm, between 0 μm and 20 μm, between 0 μm and 10 μm, between 0 μm and 5 μm, about 4 μm, about 3 μm, about 2 μm, about 1 μm, or 0 μm.

The stiffness-matched device disclosed herein may be covered in its entirety or in part with a bioactive layer based on calcium phosphate or another osseointegration-enhancing layer. These coatings, whether osseointegrated or not, are also useful as a time-sensitive way of ensuring when the device is exposed to body fluids. For example, calcium phosphate and hydroxyapatite coatings can be produced by a sol gel technique to control the timing of a resorbable Mg alloy device. As long as this coating is intact, the Mg alloy portion of the device does not degrade (i.e., does not lose mass or strength) as it is not exposed to body fluid until that coating resorbs to the point where it is breached. The loss of mechanical properties and mass begins after a pre-determined (i.e., well-characterized) period of time during which the coating degrades at a predictable rate. Thus, coating materials may be used to provide a time-certain commencement of the loss of mechanical properties and mass in the resorbable component of a bulk (i.e., two or more functional components) skeletal reconstruction device. Implant coatings may also be functionalized with cells or carriers of drugs, antibiotics, or other bioactive substances. Similarly, these bioactive materials could be conjugated to the coating's surface.

### Examples of Stiffness-Matched Devices with Non-Resorbable and Resorbable Components

The stiffness-matched devices disclosed herein may be used for bone graft fixation, replacement, and/or abutment. The stiffness-matched device disclosed herein includes a non-resorbable portion made of a non-resorbable biocompatible material. The stiffness-matched device disclosed herein includes a resorbable portion made of a resorbable biocompatible material joined to the non-resorbable portion, wherein the resorbable portion is resorbed in a pre-determined timing such that mechanical properties of the device are modulated to avoid stress shielding of a bone and to control stress concentrations in the device below a predetermined level to avoid failure of the device.

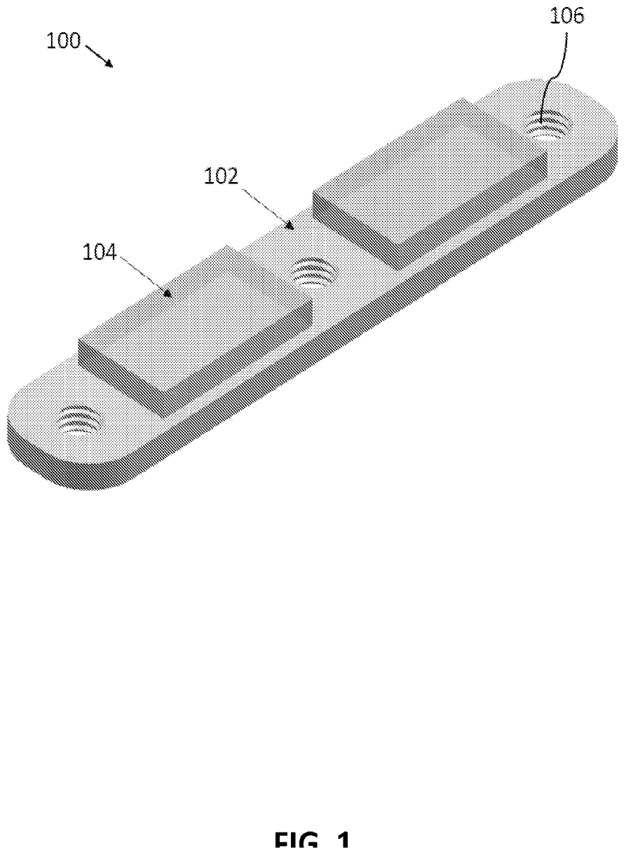

FIG. 1 shows an example of a stiffness-matched device 100 having a solid layered structure. The stiffness-matched device 100 includes a non-resorbable portion 102 and a resorbable portion 104. The non-resorbable portion 102 is made of non-resorbable biocompatible material(s) and forms the body part of the stiffness-matched device 100. The resorbable portion 104 is made of bioresorbable material(s) and is disposed on the non-resorbable 102. The non-resorbable and resorbable portions 102 and 104 are joined by any suitable method or methods, such as press fitting, welding, explosive joining, impact welding including: vaporizing foil actuator welding, magnetic impulse welding, and laser driven impact welding, pulsed and continuous wave laser welding, electron beam welding, friction welding, ultrasonic welding, ultrasonic additive manufacturing, cold spray, casting, infusion, jetting, brazing, soldering, diffusion joining, transient liquid bonding or a variety of additive manufacturing technologies. The joining method is designed to significantly reduce or eliminate intermediate phase formation between the non-resorbable and resorbable portions 102 and 104. The joining method is also designed to minimize or eliminate degrading the properties (i.e., so that the device is no longer useful) of the non-resorbable and resorbable portions of 102 and 104 through use of a joining process.

The stiffness-matched device 100 may include one or more screw threads 106 in the non-resorbable portion 102. The one or more screw threads 106 are through the body of the non-resorbable portion 102 and are configured to allow implanting the stiffness-matched device 100 to a bone. The portions of the non-resorbable portion 102 surrounding the one or more screw threads 106 are relatively or substantially flat and plate-like (e.g., as compared to a wire-like device or screw-only device). This configuration may help create surfaces that are flush to the bone when the stiffness-matched device 100 is implanted. Having these plate-like regions at or near the screws (near the one or more threads 106) may increase the strength and stiffness of the stiffness-matched device 100.

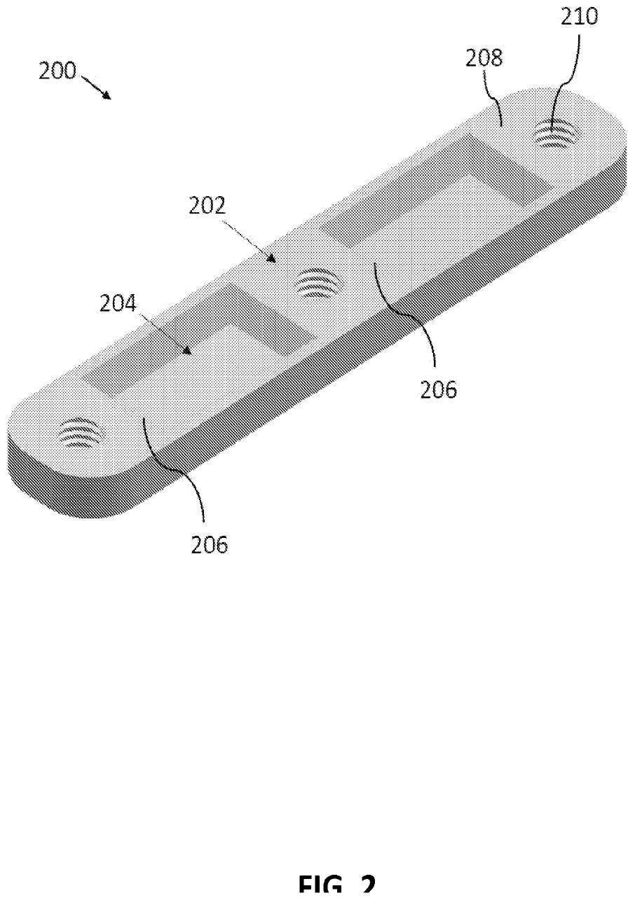

FIG. 2 shows an example of stiffness-matched device 200 having a solid layered structure. The stiffness-matched device 200 includes a non-resorbable portion 202 and a resorbable portion 204. The non-resorbable portion 202 is made of non-resorbable biocompatible material(s) and forms the body part of the stiffness-matched device 100. The resorbable portion 204 is made of bioresorbable material(s). The non-resorbable portion 202 includes one or more sockets or recessed portions 206 that are configured to receive the resorbable portion 204. The non-resorbable and resorbable portions 202 and 204 are joined by any suitable methods, such as press fitting, welding, explosive joining, impact welding including: vaporizing foil actuator welding, magnetic impulse welding, and laser driven impact welding, pulsed and continuous wave laser welding, electron beam welding, friction welding, ultrasonic welding, ultrasonic additive manufacturing, cold spray, casting, infusion, jetting, brazing, soldering, diffusion joining, transient liquid bonding or a variety of additive manufacturing technologies. The joining method is designed to significantly reduce or eliminate intermediate phase formation between the non-resorbable and resorbable portions 202 and 204. The joining method is also designed to minimize or eliminate degrading the properties of the non-resorbable and resorbable portions of 202 and 204.

In the illustrated embodiment, the resorbable portion 204 is substantially flush with surface 208 of the stiffness-matched device 200. The resorbable portion 204 is housed within the non-resorbable portion 202. In contrast, in the stiffness-matched device 100, a portion of the resorbable portion 104 is outside of the non-resorbable portion 102. In other embodiments, the resorbable portion 204 may be slightly protruding out or recessed in with respect to surface 208.

The stiffness-matched device 200 may include one or more screw threads 210 in the non-resorbable portion 202. The one or more screw threads 210 are through the body of the non-resorbable portion 202 and are configured to allow implanting the stiffness-matched device 200 to a bone. The portions of the non-resorbable portion 202 surrounding the one or more screw threads 210 are relatively or substantially flat and plate-like. This configuration may help create surfaces that are flush to the bone when the stiffness-matched device 200 is implanted. Having these plate-like regions at or near the screws (near the one or more threads 210) may increase the strength and stiffness of the stiffness-matched device 200.

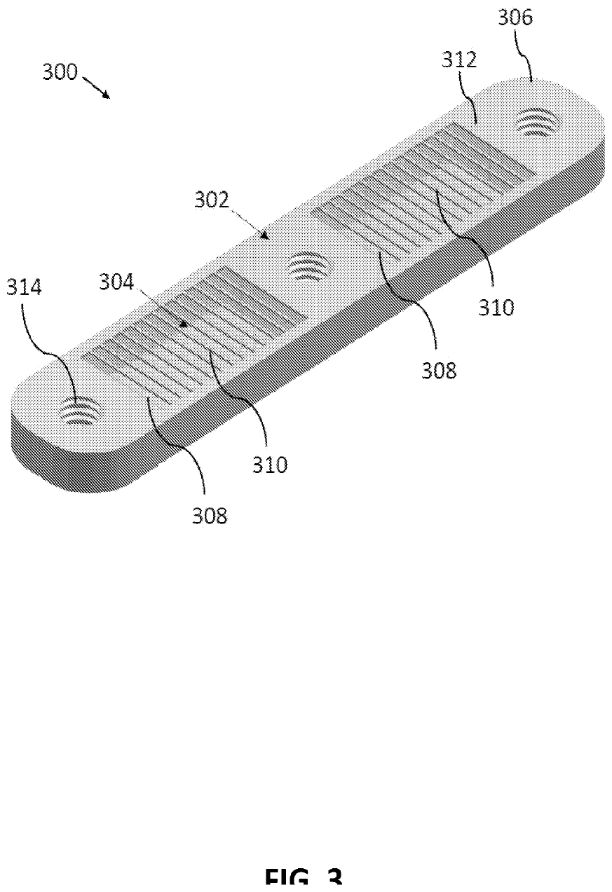

FIG. 3 shows an example stiffness-matched device 300 having a non-resorbable portion 302 made of non-resorbable biocompatible material(s) and a resorbable portion 304 made of bioresorbable material(s). The non-resorbable portion 302 includes a body 306, one or more sockets or recessed portions 308, and one or more cages 310. The one or more sockets or recessed portions 308 are configured to receive the resorbable portion 304. The one or more cages 310 are configured to at least partially close or constrain the resorbable portion 304 inside the one or more sockets or recessed portions 308. The resorbable portion 304 is housed by slats made of the non-resorbable portion 302 to prevent dislodging of large pieces of resorbing materials. The one or more cages 310 are substantially flush with a surface 312 of the stiffness-matched device 300. The non-resorbable and resorbable portions 302 and 304 are joined by any suitable methods, such as press fitting, welding, explosive joining, impact welding including: vaporizing foil actuator welding, magnetic impulse welding, and laser driven impact welding, pulsed and continuous wave laser welding, electron beam welding, friction welding, ultrasonic welding, ultrasonic additive manufacturing, cold spray, casting, infusion, jetting, brazing, soldering, diffusion joining, transient liquid bonding or a variety of additive manufacturing technologies. The joining method is designed to significantly reduce or eliminate intermediate phase formation between the non-resorbable and resorbable portions 302 and 304. The joining method is also designed to minimize or eliminate degrading the properties of the non-resorbable and resorbable portions of 302 and 304.

The stiffness-matched device 300 may include one or more screw threads 314 in the non-resorbable portion 302. The one or more screw threads 314 are through the body of the non-resorbable portion 302 and are configured to allow implanting the stiffness-matched device 300 to a bone. The portions of the non-resorbable portion 302 surrounding the one or more screw threads 314 are relatively or substantially flat and plate-like. This configuration may help create surfaces that are flush to the bone when the stiffness-matched device 300 is implanted. Having these plate-like regions at or near the screws (near the one or more threads 314) may increase the strength and stiffness of the stiffness-matched device 300.

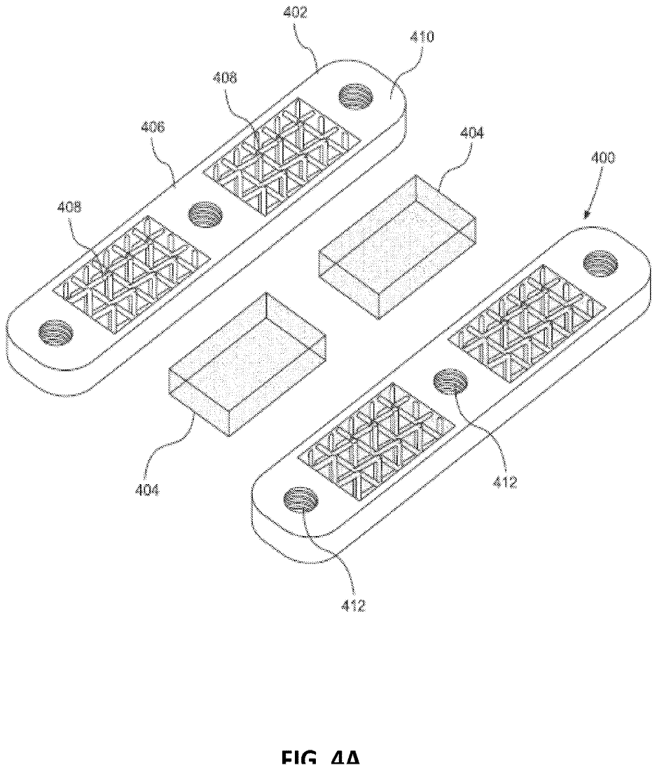

FIGS. 4A (perspective view) and 4B (side view) show an example stiffness-matched device 400 having a non-resorbable portion 402 made of non-resorbable biocompatible material(s) and a resorbable portion 404 made of bioresorbable material(s). The non-resorbable portion 402 includes a solid body 406 and one or more porous portions 408. The resorbable portion 404 is jointed to the one or more porous portions 408. The one or more porous portions 408 may contain pores (e.g., 10 volume % (vol. %) to 90 vol. % porosity, 10 vol. % to 80 vol. % porosity, 10 vol. % to 70 vol. % porosity, 10 vol. % to 60 vol. % porosity, 10 vol. % to 50 vol. % porosity, etc.) and the resorbable portion 404 fills the pores. The resorbable portion 404 is housed within the non-resorbable portion 402. The resorbable portion 404 has a scaffold or porous structured with planned porosity. pores (e.g., 10 volume % (vol. %) to 90 vol. % porosity, 10 vol. % to 80 vol. % porosity, 10 vol. % to 70 vol. % porosity, 10 vol. % to 60 vol. % porosity, 10 vol. % to 50 vol. % porosity, etc.). In the illustrated embodiment, the resorbable portion 404 filled the one or more porous portions 408 are substantially flush with a surface 410 of the non-resorbable portion 402. In other embodiments, the resorbable portion 404 filled one or more porous portions 408 may be slightly protruding out or recessed in with respect to the surface 410. The one or more porous portions 408 may be formed of any suitable pore morphologies/structures. The pore structure may help contain or cage the resorbable portion 404 in the one or more porous portions 408. The non-resorbable and resorbable portions 402 and 404 are joined by any suitable methods, such as press fitting, welding, sintering, explosive joining, impact welding including: vaporizing foil actuator welding, magnetic impulse welding, and laser driven impact welding, pulsed and continuous wave laser welding, electron beam welding, friction welding, ultrasonic welding, ultrasonic additive manufacturing, cold spray, casting, infusion, jetting, brazing, soldering, diffusion joining, transient liquid bonding or a variety of additive manufacturing technologies. The joining method is designed to significantly reduce or eliminate intermediate phase formation between the non-resorbable and resorbable portions 402 and 404. The joining method is also designed to minimize or eliminate the joining causing degrading the properties of the non-resorbable and resorbable portions of 402 and 404 below that needed for the device to function as intended.

The stiffness-matched device 400 may include one or more screw threads 412 in the non-resorbable portion 402. The one or more screw threads 412 are through the body of the non-resorbable portion 402 and are configured to allow implanting the stiffness-matched device 400 to a bone. The portions of the non-resorbable portion 402 surrounding the one or more screw threads 412 are relatively or substantially flat and plate-like. This configuration may help create surfaces that are flush to the bone when the stiffness-matched device 400 is implanted. Having these plate-like regions at or near the screws (near the one or more threads 412) may increase the strength and stiffness of the stiffness-matched device 400.

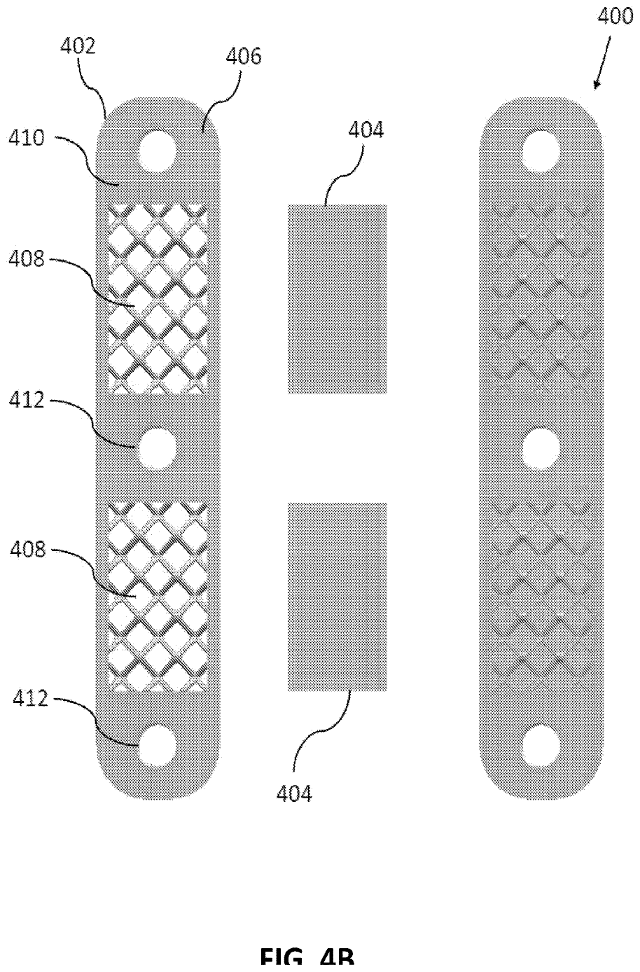

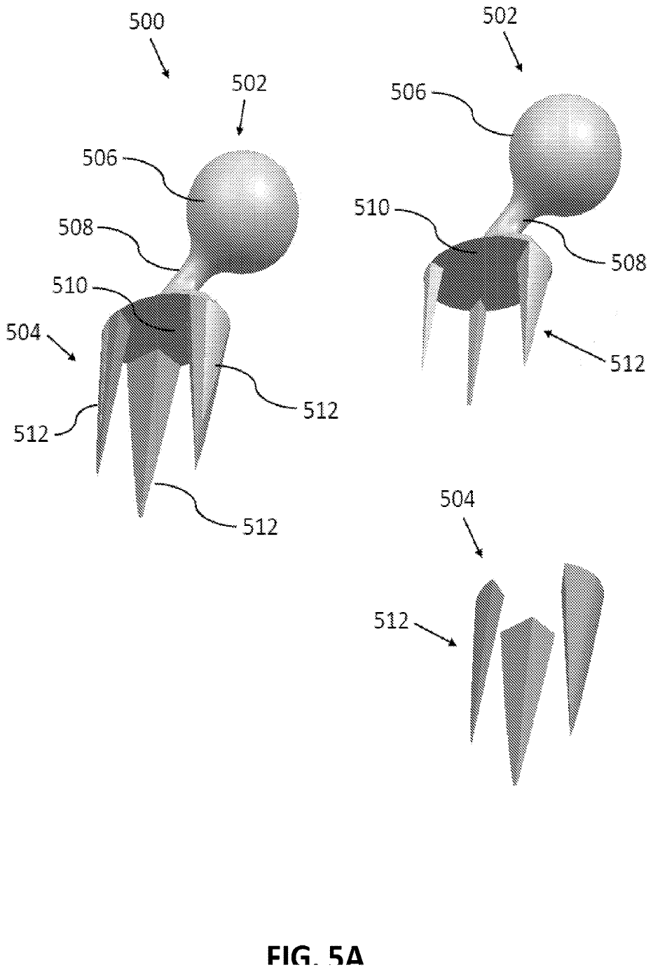

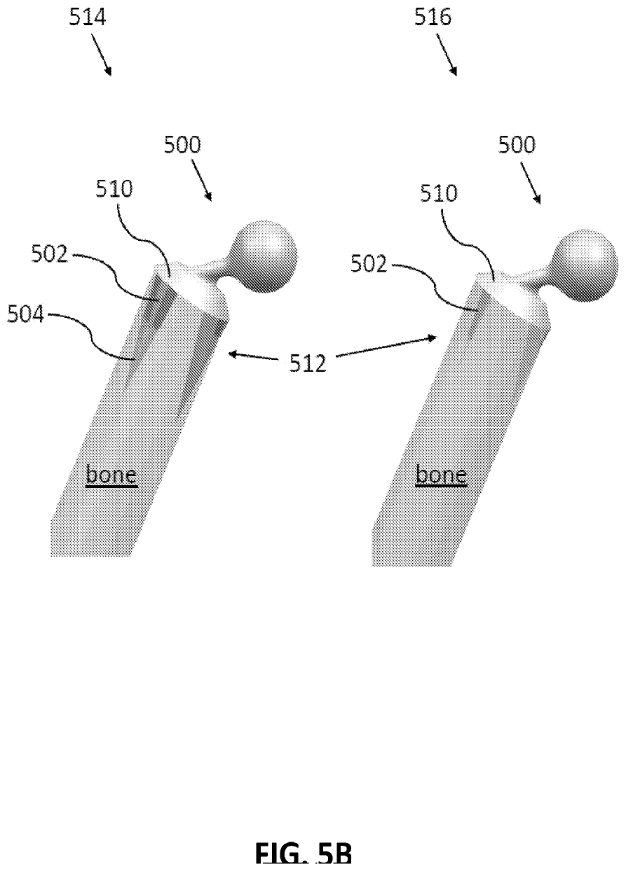

FIGS. 5A-5C shows an example of stiffness-matched device 500 configured to have a “fluted cuff” shape. The stiffness-matched device 500 includes a non-resorbable portion 502 and a resorbable portion 504. The non-resorbable portion 502 is made of non-resorbable biocompatible material(s) and forms the body part of the stiffness-matched device 500. The resorbable portion 504 is made of bioresorbable material(s).

The stiffness-matched device 500 includes a head 506, a neck 508, a base 510, and one or more protrusions 512. The head 506, the neck 508, and a part of the one more protrusions 512 are the non-resorbable portion 502. The resorbable portion 504 is shaped to be joined to the non-resorbable portion 502 of the one or more protrusions 510. The one or more protrusions 512 may include finger-like or strut-like non-resorbable portion 502 and also finger-like or strut-like resorbable portion 504 that goes along the length of the finger-like or strut-like non-resorbable portion 502. The one or more protrusions 512 are configured to attach to a bone (e.g., as in hip, knee, shoulder, spine, or skull replacement, or percutaneous implants). These protrusions 512 or fingers may be threaded (e.g., cylindrical or semicylindrical screw or screw-like), textured, or porous to provide better attachment and load transfer.

The non-resorbable and resorbable portions 502 and 504 are joined by any suitable methods, such as press fitting, welding, explosive joining, impact welding including: vaporizing foil actuator welding, magnetic impulse welding, and laser driven impact welding, pulsed and continuous wave laser welding, electron beam welding, friction welding, ultrasonic welding, ultrasonic additive manufacturing, cold spray, casting, infusion, jetting, brazing, soldering, diffusion joining, transient liquid bonding or a variety of additive manufacturing technologies. The joining method is designed to significantly reduce or eliminate intermediate phase formation between the non-resorbable and resorbable portions 502 and 504. The joining method is also designed to minimize or eliminate degrading the properties of the non-resorbable and resorbable portions of 502 and 504.

In application (e.g., replacement or percutaneous applications), the stiffness-matched device 500 sits inside or on top of a bone as shown in FIG. 5B. In an implantation phase 514, the one or more protrusions 512 are inserted into the bone and the base 510 is abut a terminal end or a rough surface of the bone. For example, the stiffness-matched device 500 may be a proximal hip replacement device that translates directly to a cortical bone. Upon implantation, the resorbable portion 504 is being resorbed such that in an after-healing phase 516, the stiffness-matched device 500 is integrated with the bone growing between the remining non-resorbable portion 502 (e.g., the one or more protrusions 512). In applications of percutaneous implants, an “offloading” or load-bearing would occur in the stiffness-matched device 500 with the resorbable one non-resorbable fingers (e.g., the one or more protrusions 512) in the cortex, where the resorbable portion 504 promotes bone ingrowth as the resorbable portion 504 is resorbed. The resorbable portion 504 may be designed with texture and/or porosity to assist the resorption.

The stiffness-matched device 500 may have any shapes (e.g., cylindrical, cone, etc.) and dimensions suitable for replacement or percutaneous implant applications. FIG. 5C shows another example of the stiffness-matched device 500. In this example, the stiffness-matched device 500 includes one protrusion 512 having a characteristic width 518 that has a wider end 520 near the base 510 and gradually decreases to a narrower end 522. The protrusion 512 includes the non-resorbable portion 502 that includes grooves 524 along the length of the protrusion 512. The protrusion 512 further includes the resorbable portion 504 that is shaped to be joined to the non-resorbable portion 502 in the grooves 524 (e.g., at least partially fills the grooves 524). The stiffness-matched device 500 discussed above may have different shapes and dimensions, and the bulkier device (e.g., FIG. 5C) may be more helpful for an older patient.

In the stiffness-matched devices disclosed herein, the non-resorbable biocompatible material(s) may include metal, metal alloy, polymer, ceramic, composite, or a combination thereof. In one example, the non-resorbable biocompatible materials include nickel-titanium (NiTi) alloys, or any other shape memory alloy.

The bioresorbable material(s) may include metal, metal alloy, polymer, ceramic, composite, or a combination thereof. In one example, the resorbable materials include magnesium (Mg) alloys (e.g., WE43, Mg—Zn—Ca—Mn alloy) and other bioresorbable metals—zinc or ion and their alloys.

The joining method is designed to eliminate, prevent, or significantly reduce intermediate phase formation between the non-resorbable and resorbable portions. The joining method is also designed to minimize or eliminate degrading the properties of the non-resorbable and resorbable portions.

### Fabrication

The stiffness-matched devices disclosed herein are fabricated via various approaches and techniques as discussed below. For example, the joining technologies including mechanical connections, press fitting, welding, explosive joining, impact welding including: vaporizing foil actuator welding, magnetic impulse welding, and laser driven impact welding, pulsed and continuous wave laser welding, electron beam welding, friction welding, ultrasonic welding, ultrasonic additive manufacturing, cold spray, casting, infusion, jetting, brazing, soldering, diffusion joining, transient liquid bonding or a variety of additive manufacturing technologies, including manufacturing of the components separately, or during one manufacturing process (multi-material manufacturing), may be applied to join the non-resorbable and resorbable portions together. In some embodiments, the non-resorbable and resorbable portions of the stiffness-matched devices are joined or attached to one another via methods that significantly avoid or reduce the formation of detrimental brittle phases, and limit any changes to the microstructure and chemistry of the resorbable and non-resorbable alloys. Both the resorbable and non-resorbable portions are designed to offer appropriate stiffness to facilitate skeletal healing and regeneration.

The stiffness-matched devices disclosed herein may also be configured to provide personalization to allow for a flush-fitting application to a boney surface (e.g., the device is as flush as possible, especially in a region of contact and/or screw fixation). The stiffness-matched devices disclosed herein may be made by fabricating the non-resorbable portion with personalized (e.g., patient-specific) shapes and dimensions. In cases that the non-resorbable portion includes one or more porous portions, the porous portions are fabricated with designed characteristics (e.g., designed pore morphology, porosity, etc.) for stiffness-modulating purposes. The one or more porous portions are also fabricated with designed dimensions, and possibly algorithmic definition of the pore geometry to achieve desired mechanical properties, ready for joining with the body (e.g., non-porous portion) of the non-resorbable portion via any suitable methods, such as laser welding.

In the stiffness-matched devices disclosed herein, the effective load bearing during fixation is achieved by tailoring the density of the resorbable portion as part of the overall structure. The density of the resorbable portion is changeable through designing (as opposed to random and impermeable such as using porogens or incomplete forming that results in non-dense parts) one or more porous portions of the resorbable and/or non-resorbable portion with desired pore characteristics and geometry (e.g., e.g., pore morphology, pore size and distribution, uniform, gradient, and/or adaptive). The density of the resorbable portion is also changeable through designing the pore characteristics and geometry (e.g., pore morphology, pore size and distribution, uniform, gradient, and/or adaptive) of the resorbable portion.

Tuning the density of the resorbable portion through planned, perhaps 3D printed porosity, allows sufficient predictable integration of the two components (e.g., the non-resorbable and resorbable portions) and modeling of the immobilization. This in turn allows more effective choice of shape and location of the device implant on the skeleton. The mechanical, shape, and attachment site may be designed together in an interactive model of bone fracture or graft immobilization. The planned start and duration of resorption can also be used in the presurgical planning of the fixation strategy. Pre-planning allows personalization during fabrication of the devices. Shape adjustment of the stiffness-matched devices disclosed herein may be done manually, e.g., by bending the non-resorbable portion of the device, or may be achieved via automated mechanical deformation or other shape altering methods that can be used to affect the desired mechanical performance.

### Coatings and Polishing

Coatings may be applied to the stiffness-matched devices disclosed herein. The coatings may be made of bioresorbable material(s), including but not limited to, magnesium (Mg) alloys (e.g., WE43, Mg—Zn—Ca—Mn alloy), and other bioresorbable metals-zinc or ion, and their alloys. A coating may be applied to coat the whole or at least a portion of the stiffness-matched devices disclosed herein. As the coating is resorbed, it may be desirable to leave a porous region into which new bone could be encouraged to develop (e.g., following tissue seeding and provision of cytokines) or grow.

A coating may be applied to the stiffness-matched devices disclosed herein to seal the resorbable portion. By timing the resorption of the coating, as the coating is resorbed the underlying resorbable portion is exposed at a time when the fixation device is no longer needed (e.g., when sufficient healing has occurred) such that the resorbable portion is being resorbed during or post healing.

The surface of the stiffness-matched devices may be polished via a suitable polishing technique (e.g., manual polishing, electrochemical polishing, etc.) to help prevent crack formation and reduce risk of early loss of mechanical properties.

### Virtual Surgical Planning

Skeletal reconstruction devices are often held in place by standard or locking screws of the same or a similar material. The design, location, and material choice of the bone screw may affect the performance of the stiffness-matched devices disclosed herein. Well known techniques for locking screws may benefit from careful changes in implantation angulation from screw to screw (i.e., having different entry angles into the cortical bone as seen in plates that wrap around ribs with the screws entering the rib at different angles.), allowing them to tighten in response to adjacent load rather than to loosen. Similarly, improved elasticity may allow engagement of whole plate rather than highly strong and stiff plate localizing the load and possibly overwhelming the local component. Similarly, local strength in the region of a screw can be enhanced by planning bicortical rather than unicortical placement. Personalization of the shape and mechanical properties of the skeletal reconstruction device (e.g., the stiffness-matched device disclosed herein) may also help create surfaces that are flush to the bone. Avoiding manual bending in crimp zones can avoid work-hardening seen in off-the-shelf plates with limited zones for manual bending. Limited bending zones may also prevent surface flushness compared to 3D printing. Manual bending of plates without virtual surgical planning (VSP) information on the effect of plate location, geometry, or material must rely heavily on surgeon experience and intuition rather than post-operative performance modeling. Having these plate-like regions at or near screws may increase the strength and stiffness of the device.

Unplanned gaps between the device and the bone may isolate load in the device. Therefore, when implanting the stiffness-matched devices disclosed herein, unplanned gaps between the device and the bone are minimized or avoided to improve enhancement of contact. Moreover, location can allow stiffness matching to the job of providing fixation of graft and host segments so that loading in the unhealed configuration can bring about compression of unhealed segments whereas one can choose plate location, geometry, and material so that healed segments will re-establish or establish a new sufficient and normal loading patterns that maintain bone density through healthy remodeling processes. The post-healing resorption of key segments of the plate can also help ensure that the fixation plate does not interrupt normal, bone preserving, loading patterns.

As an example, the components of the stiffness-matched devices disclosed herein may be made either by a centralized company or a Point-of-Care manufacturing (POCM) company. Use of POCM technologies could make these personalized parts available to those who have suffered significant trauma or will have a skeletal cancer resection due to cancer sufficiently quickly to be of use within the time frame needed for a definitive post-trauma surgery or to remove a malignant tumor.

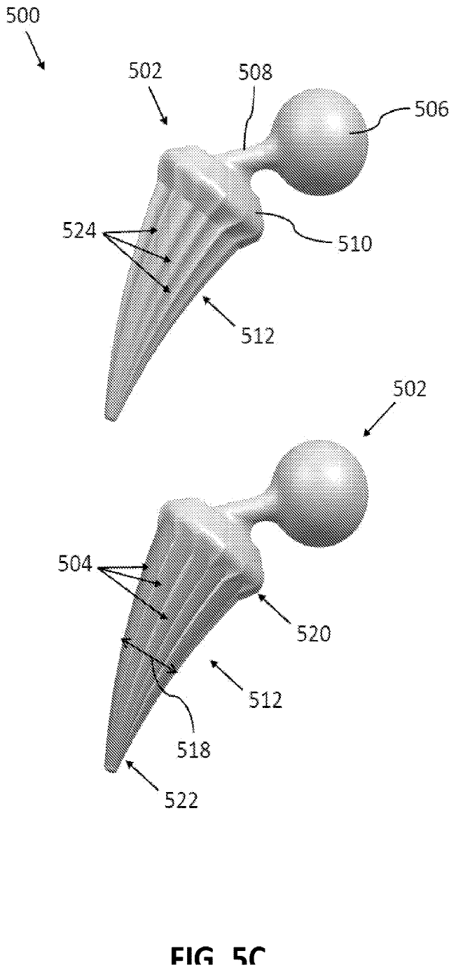

FIG. 6 shows an example process 600 of making and using the stiffness-matched devices disclosed herein. The process 600 may include obtaining a non-resorbable portion and a resorbable portion for a stiffness-matched device disclosed herein (step 602). The process 600 may include joining the non-resorbable portion and the resorbable portion to form the stiffness-matched device disclosed herein (step 604). The process 600 may optionally include coating and/or polishing the stiffness-matched device disclosed herein (step 606). The process 600 may include implanting the stiffness-matched device disclosed herein (step 608).

In step 602, the non-resorbable and resorbable portions are fabricated or obtained. This step may be accomplished with a high level of personalization. For example, the shape and dimensions of the non-resorbable and resorbable portions are designed and made to help create surfaces that are flush to the bone of a patient (this can be done in a patient-specific or non-patient-specific way). For example, the material choices for the non-resorbable and resorbable portions are selected according to the needs of the patient with mechanical properties that are matched according to the bone healing progress.

In step 604, any suitable joining methods may be used to join the non-resorbable and the resorbable portions, for example, press fitting, welding, explosive joining, impact welding including: vaporizing foil actuator welding, magnetic impulse welding, and laser driven impact welding, pulsed and continuous wave laser welding, electron beam welding, friction welding, ultrasonic welding, ultrasonic additive manufacturing, cold spray, casting, infusion, jetting, brazing, soldering, diffusion joining, transient liquid bonding or a variety of additive manufacturing technologies, as discussed above. The joining method may be designed/selected to significantly avoid or reduce the formation of detrimental brittle phases or damage (heat damage) to the material(s) that form the non-resorbable portions (non-resorbable biocompatible metal or alloy).

The joining method is designed to significantly reduce or eliminate undesirable intermediate phase formation between the non-resorbable and resorbable portions.

The joining method is designed to minimize or eliminate degrading the properties of the non-resorbable and resorbable portions.

In some embodiments, steps 602 and 604 may be combined as one step or done simultaneously. For example, the non-resorbable and resorbable portions may be manufactured and joined via additive manufacturing. Additive manufacturing of multi-materials enables both alloys (the resorbable and non-resorbable materials) to be fabricated and joined together during fabrication. In some embodiments, steps 602 and 604 may occur simultaneously. For example, the components (e.g., the resorbable and non-resorbable portions) may be produced simultaneously, such as layer by layer, via additive manufacturing, and the components may be joined via the additive manufacturing process (as opposed to joining in a subsequent step).

In step 606, coating and/or polishing may be optionally applied to the stiffness-matched device as discussed above. Specifically, the coating may be applied in a way to time resorption of the resorbable portion with the bone healing progress.

In step 608, the stiffness-matched device disclosed herein may be implanted via screws (through the one or more threads) to be fixed to the bone. The geometries/shapes of the stiffness-matched device components are designed to eliminate or significantly minimize unplanned gaps between the device and the bone to improve enhancement of contact.

### EXAMPLES

Below are non-limiting examples for certain aspects of the present embodiments disclosed herein.

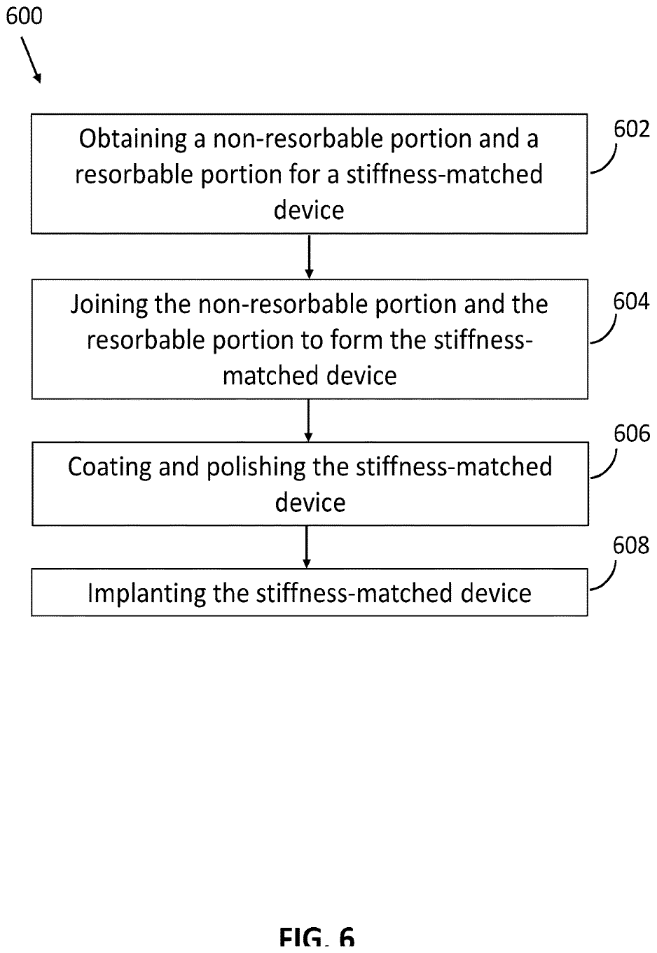

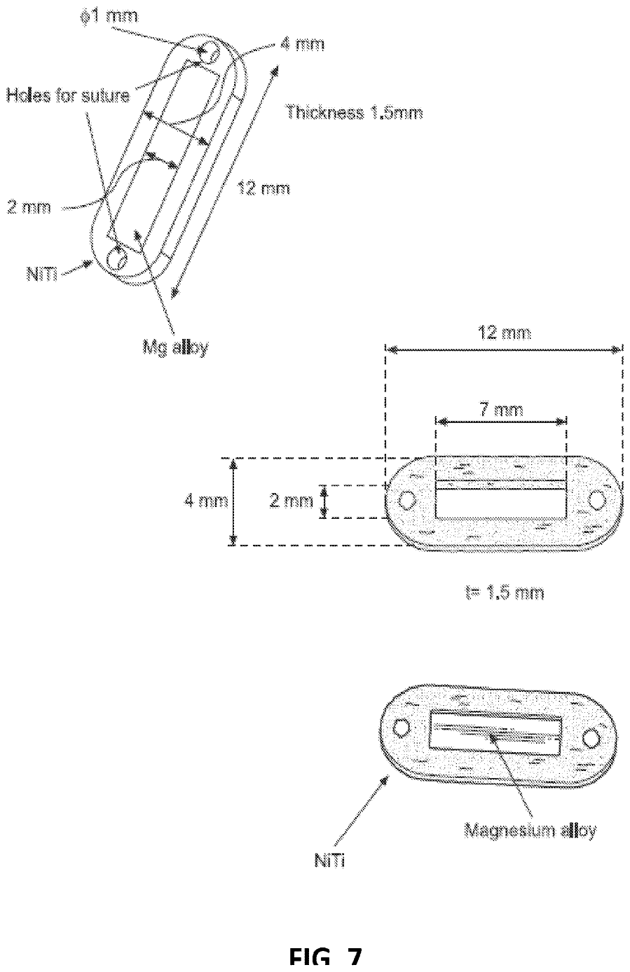

FIG. 7 and FIG. 8 each show a non-limiting example stiffness-matched device for a rat model study. In both cases, the magnesium (e.g., Mg alloy) resorbable portion and the non-resorbable portion (e.g., NiTi alloy) are made via additive manufacturing and press-fitted to join one another.

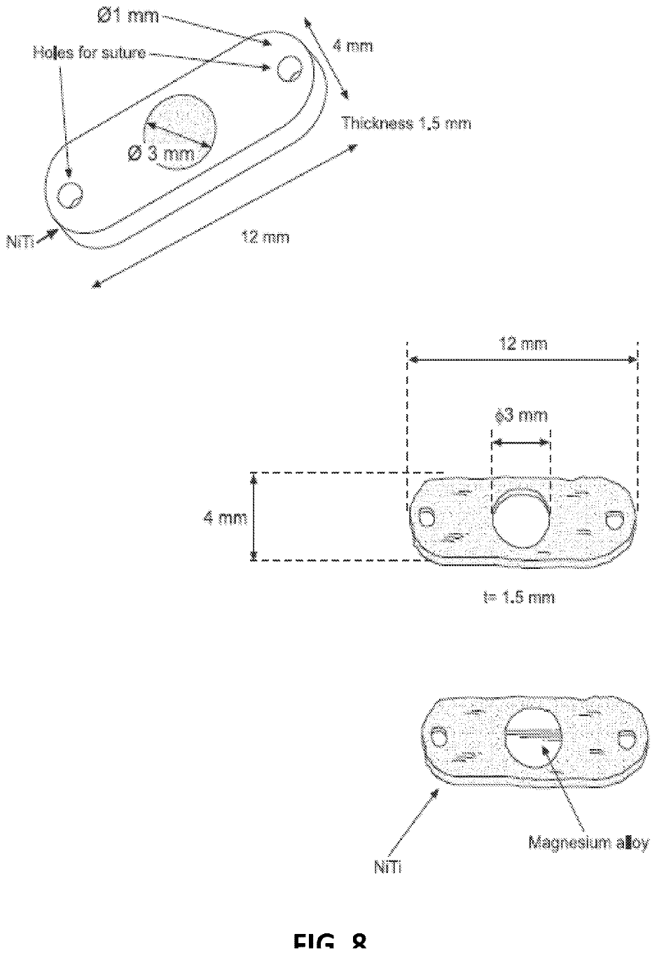

FIG. 9 shows an example of virtual surgical planning of the stiffness-matched device in a rat model sample.

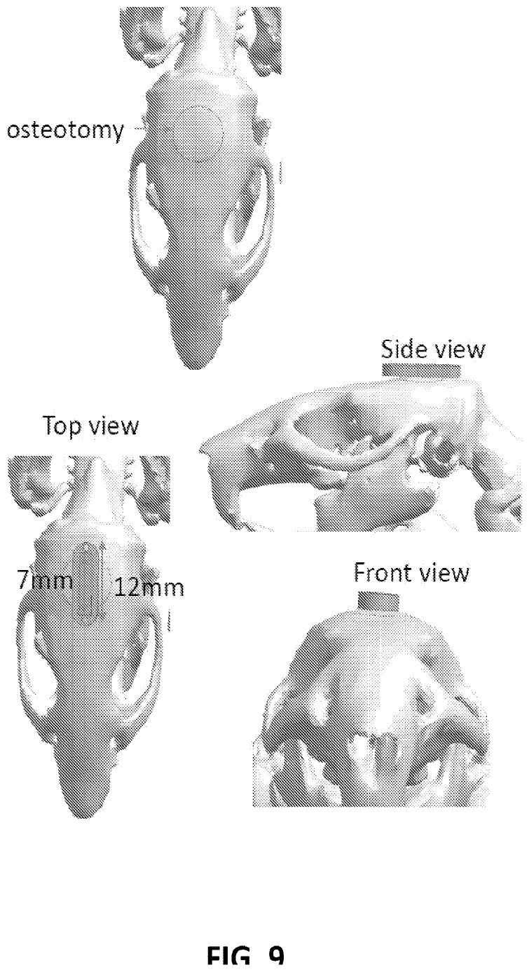

FIG. 10 shows example NiTi gyroid structures of different porosities manufactured by laser powder bed fusion (LPBF) process. In the illustrated embodiment each of the gyroid samples is a 6×6×6 units cell and has a strut size of 0.5 millimeters (mm). From left to right, the NiTi gyroid has a pore size of 0.5 mm, 1.0 mm, 1.5 mm, and 2.0 mm, respectively.

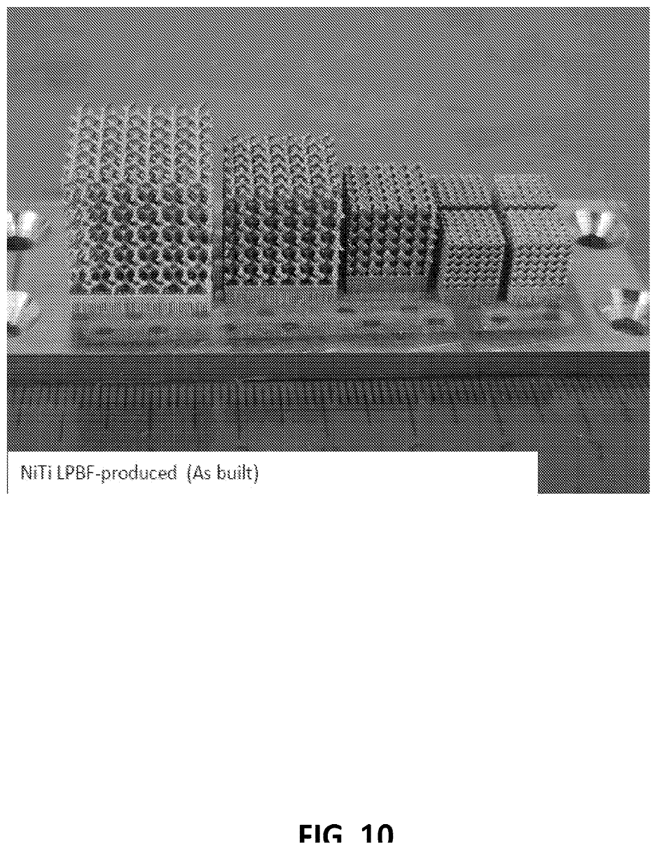

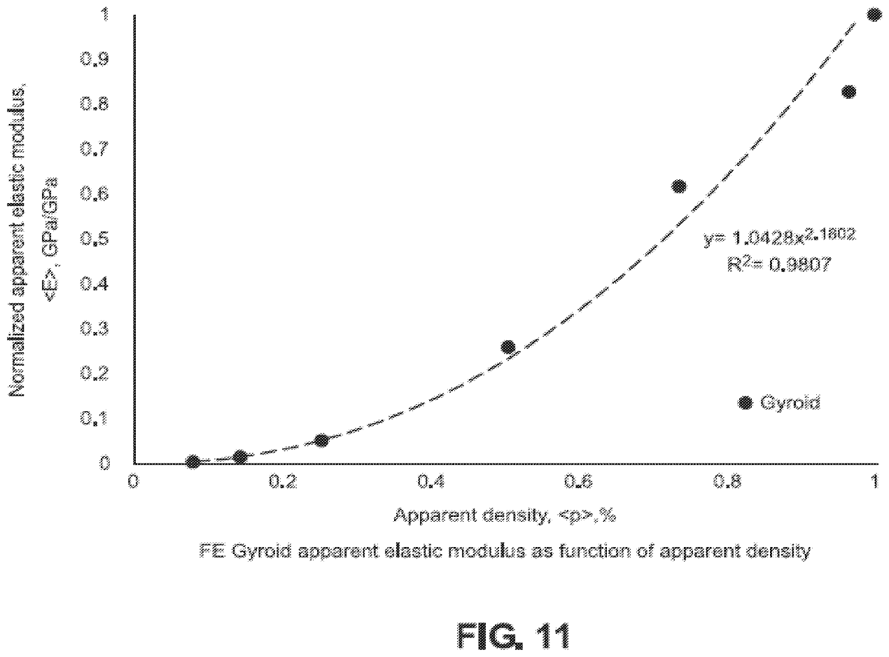

The mechanical properties of the gyroid structures are tuned by changing the porosities. For example, FIG. 11 shows an example normalized apparent elastic modulus of NiTi gyroids (GPa/GPa) as a function of apparent density (%) under compression tests. The non-resorbable porous portions of the stiffness-matched device may be manufactured by such approach to tailor the mechanical properties and thereby achieving the desired stiffness matching effects. For example, FIG. 12 shows mechanical testing results of the gyroid structures under four-point bending according to ASTM F382. The load-displacement curves of the porous samples show comparison between orthogonal strut samples and gyroid samples. The augmented view shows proof load values for sample plate made of the porous orthogonal struts and the porous gyroids. The computed mechanical properties with non-statistical differences (p>0.05) are as follows. The orthogonal structure has a bending stiffness K of 169.03±31.83 N/mm, and the gyroid structure has a K of 145.62±31.68 N/mm. Proof loads P are (obtained from the intersection of the curve with a 0.036 mm offset line) 45.91±8.86 N and 49.22±7.05 N for the orthogonal and gyroid structures, respectively. The bending structural stiffness are 274.67±51.72 kN·mm² and 236.63±51.47 kN mm² for the orthogonal and gyroid structures, respectively. The bending strength are 348.93±67.30 N·mm and 374.07±53.57 N·mm for the orthogonal and gyroid structures, respectively.

As aforementioned, the method to join the resorbable and non-resorbable portions of the stiffness-matched device is selected such that the joint region is substantially free of undesirable intermediate phase formation. The joining method is also selected to minimize or eliminate degrading the properties of the non-resorbable and resorbable portions below that needed for the device to function as intended.

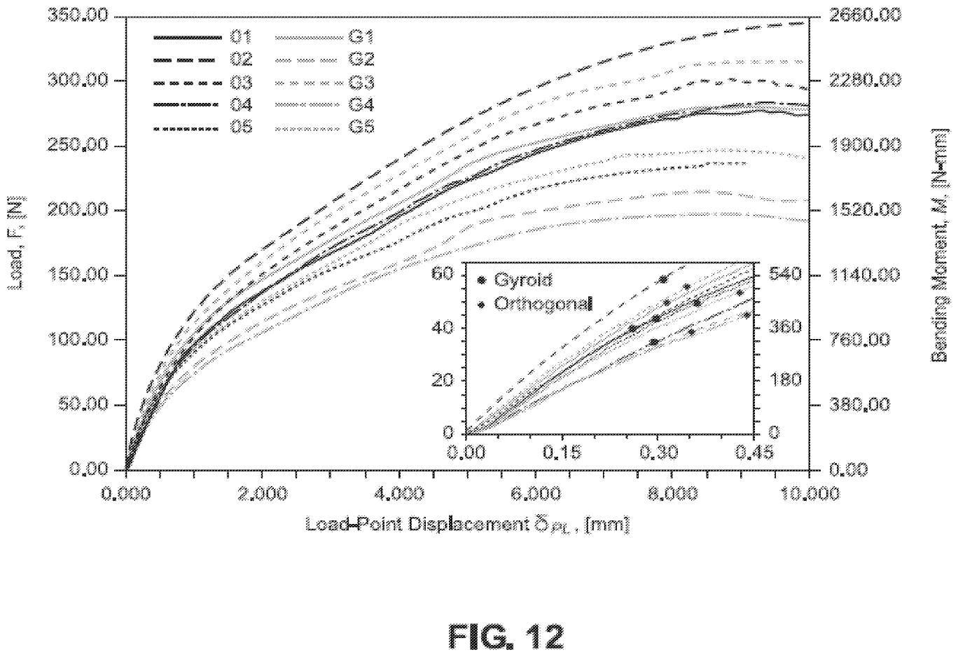

In some embodiments, it may be beneficial to form an interlayer between the non-resorbable and resorbable portions to prevent mixing/alloying of the non-resorbable and resorbable portions, and thereby significantly reduces or eliminates undesirable intermediate phase formation. For example, zinc (Zn) may be melted and poured between the resorbable (e.g., Mg alloy) portion and the non-resorbable (e.g., NiTi alloy) portion. The resorbable and non-resorbable portions are joined before Zn solidifies and forms an interlayer between the two. Once the resorbable and non-resorbable portions are joined with the Zn interlayer in between, the device may be further processed (e.g., polishing, machining, etc.) into desirable geometry and/or dimensions. FIG. 13 shows an example manufacturing method for forming a Zn interlayer between NiTi alloy and Mg alloy. Step 1 includes machining a Mg alloy into a conical shape that can be inserted into the hole located in the center of the NiTi frame. Step 2 includes melting pure Zn to 450 degrees Celsius (° C.) and then pouring it into a steel crucible heated to 450° C. Step 3 includes placing the NiTi frame on top of the molten Zn and then using an inert carbon rod or a steel rod to push the NiTi frame into the molten material, ensuring that the Zn fills the space between the holes. Step 4 includes before the molten Zn solidifies, quickly pushing the conical Mg alloy into the space between the holes, applying sufficient pressure to keep the Mg alloy from floating until the Zn solidifies. Step 5 includes polishing the Mg cone until its thickness is equal to that of the NiTi frame. In some embodiments, instead of Zn, the interlayer may be formed using other suitable biocompatible materials, such as tantalum, iron, niobium, gold, silver, platinum, palladium, iridium, aluminum, cobalt, copper, titanium, and/or their alloys or a combination thereof.

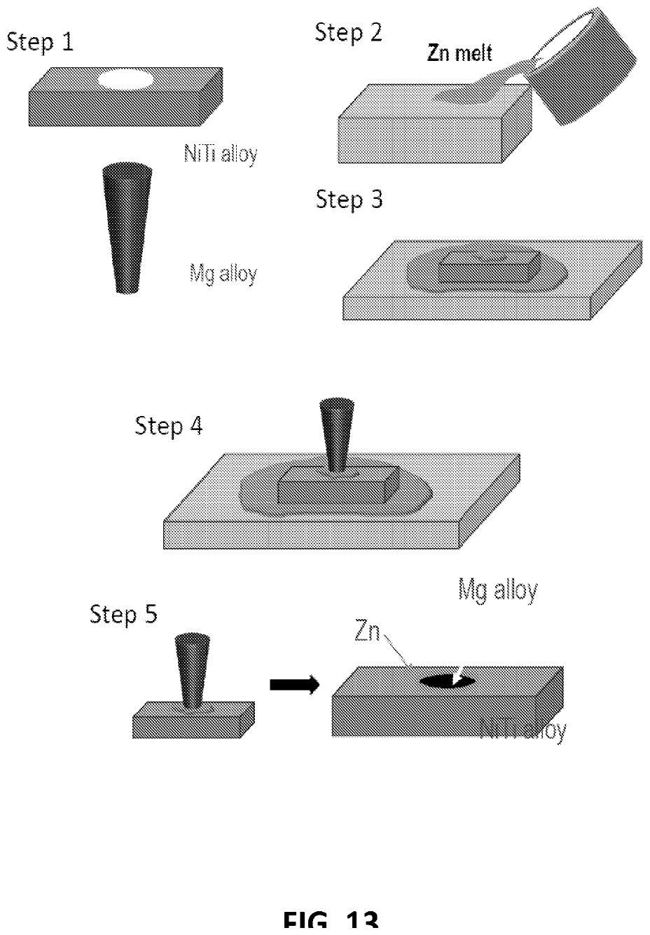

FIG. 14 shows an example virtual surgical planning (VSP) process of implanting the stiffness-matched devices disclosed herein. The VSP process includes building an anatomical model (step 1), implant designing (step 2), performing mechanical validation (step 3), and modifying/updating implant design based on mechanical validation results. For example, at least material selection, location, and/or geometry of the implant design are modified/updated based on mechanical validation results. Finally, the stiffness-matched device designed via the VSP process is manufactured (step 4) by one or more methods/processes disclosed herein.

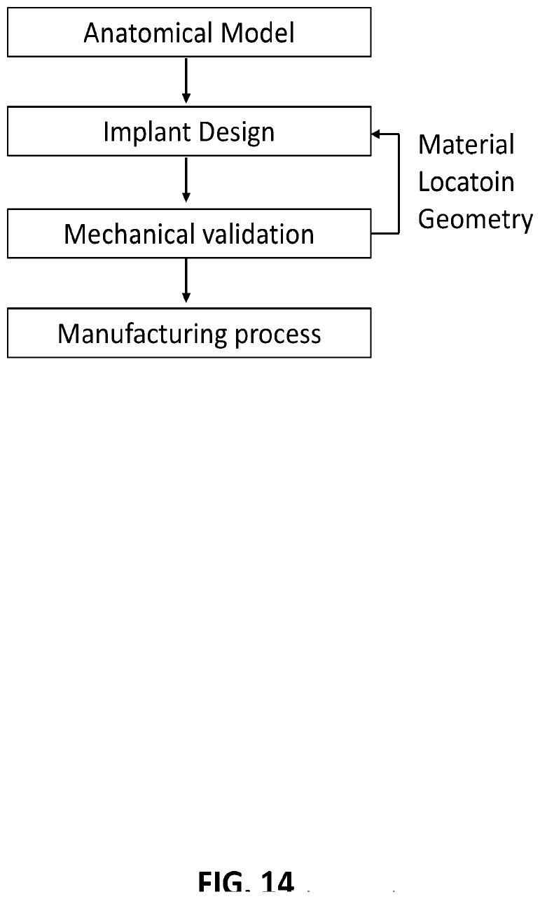

An anatomic digital model (in step 1) may be built based on medical image segmentation involving the extraction of regions of interest from 3D image data, such as from magnetic resonance imaging (MRI) or computed tomography (CT) scans. The process of implant design (step 2) may include surface detection, implant design and screw planning, and building a preliminary design. FIG. 15 shows an example workflow of implant design of a stiffness-matched bone fixation for a mandibular bone. The stiffness-matched bone fixation is designed such that after tumor resection, trauma, or inflammatory process, mandibular bone needs to be resected so the patient's aesthetics, speech, chewing, and breathing are not compromised. As a preliminary design, a solid shell and a porous core are joined together to form the preliminary device, which is then validated (step 3) via chewing modeling and finite element analysis in order to refine the design (e.g., modifying the material choices, location, and/or geometry) before manufacturing the stiffness-matched device (step 4).

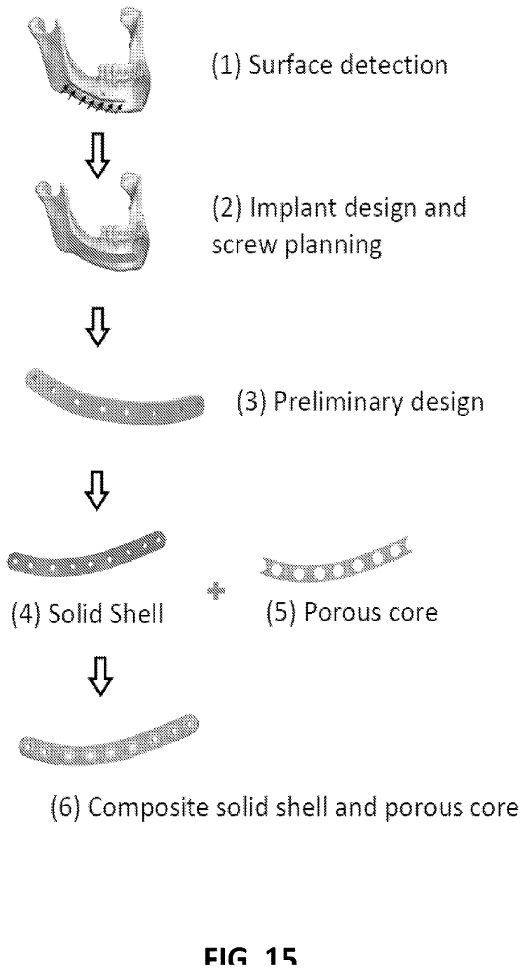

FIGS. 16-21 show example mechanical evaluation/biomedical assessments of a fixation plate for mandibular bone reconstruction. The mandibular fixation plate's developed design and verification is conducted in a VSP environment based on commercially available software for image segmentation, surgery planning, implant design and mechanical computational evaluation. The VSP process starts with medical images (i.e., CT, MRI, etc.) to obtain the anatomical component of interest. Then, it is transferred for conducting the digital osteotomy and designing the geometry and location of the implant, and then the evaluation of the design decisions under biomechanical loading. Stress and strain results give feedback to the design stage to change the geometry, location or material of the implant. Once optimized, the implant can be manufactured.

In this illustrated example, mandibular bone implants are designed for cadaveric sheep specimens. The sheep anatomical model is reconstructed from computed tomography (CT) scans with a slide thickness of 0.244 mm (machine model). DICOM images are imported to Amira software (Thermo Fisher Scientific, Waltham, MA, US). Mandibular bone is labeled by automatic and manual segmentation based on gray-scale value. The surface mesh is generated and exported as a stereolithography (STL) file.

FIG. 16 shows three implant (mandibular bone fixation plates) designs at different implant locations (“straight,” “angled,” and “warped”). In the “straight” design, the implant is located straight across the surface with both ending points at the same height. In the “angled” design, the posterior end of the plate starts high in the premolar area, without affecting teeth roots, and ends low in the anterior section of the diastema. In the “warped” design, the implant starts and ends in the same manner as in the “angled” design but wraps the mandible. While all three designs are shaped to the patient's anatomy, the first design (“straight”) is fitted to the surface, the second one (“angled”) features just bending around the bone, and the third one (“warped”) features bending and twisting around the bone.

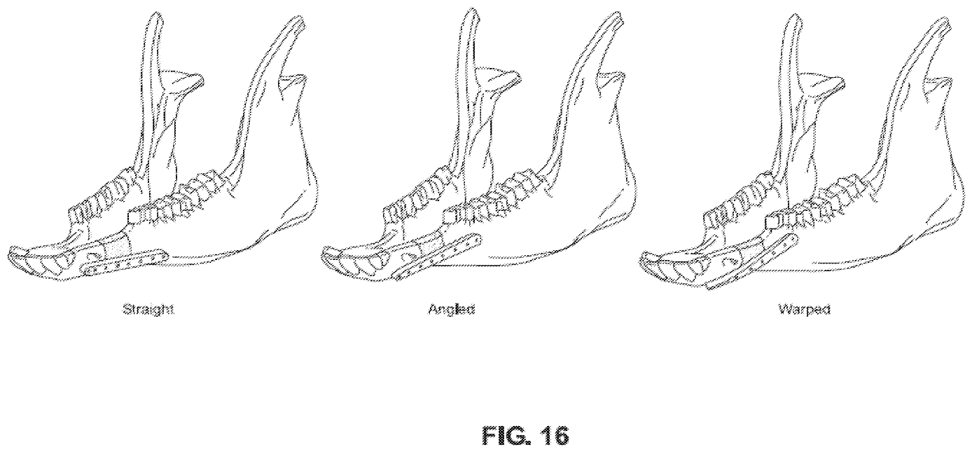

The mandibular model is imported to Geomagic Freeform (3D Systems, Rock Hill, SC, US) software for planning the virtual surgery, graft placement, implant design, and screw planning as illustrated in FIG. 17. First, an osteotomy of 15 mm in length is done virtually on the left proximal diastema, just in front of the premolars, and the resected bone section is used as the bone graft. Next, a 60 mm line, that represents the implant's center line and location, is drawn across the buccal bone surface, passing through the host mandible and the bone graft. This step ensures that the implant is flushed to the bony surface. This line is done considering the location of two screws in the grafted bone and three screws at each host mandibular side. In the second step, the width (6 mm) and the thickness (3 mm) of the implant are set. Finally, 1.9 mm diameter screws-holes are drilled into the plate. The screws'length is designed so they are locking screws and bicortical, meaning that the head of the screw is threaded and inserted into the plate, and the length of the screw crosses both cortical sections of the mandibular bone. To simplify the model, the thread and the head of the screws are not designed.

Next, the resected mandibles, graft bone, screws, and plate designs are exported to mechanical computational analysis software. The models are computationally validated under biomechanical loading. The models are imported into commercial finite element software ANSYS Workbench (ANSYS, Canonsburg, PA, US) where materials assignment, components connection, meshing, and boundary conditions are defined. Effects of varying the implant location and fixation plate material are evaluated by comparing the maximum resulting Von Mises stress on the plate for both bone conditions (before-healing and post-healing conditions) and by comparing the maximum anterior and posterior gap formation between the graft and the host mandible for the before-healing scenario.

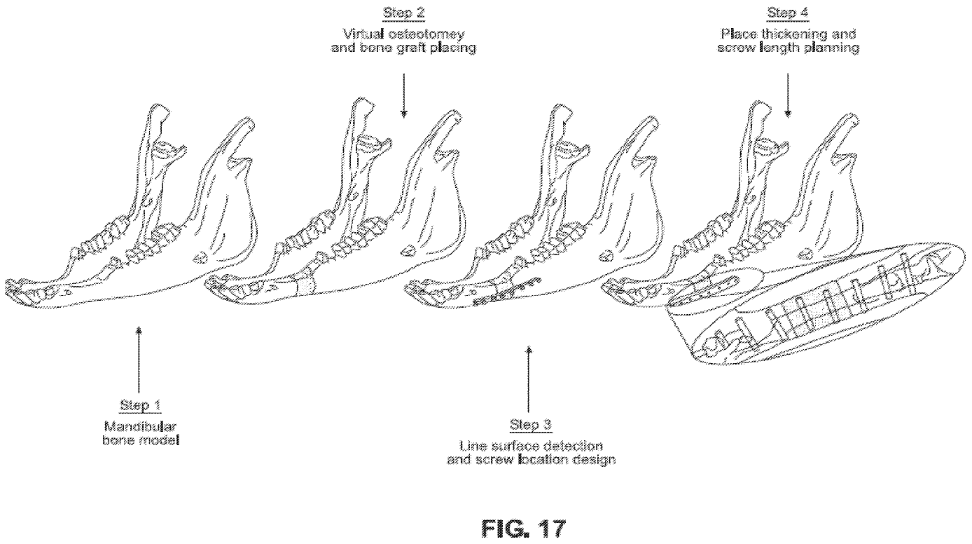

In the simulation results shown in FIGS. 18-21, the material choice of the stiffness-matched device (fixation plate) varies between Ti-6A1-4V (Ti64) alloy and NiTi alloy and the implant location varies from “straight” to “angled” to “warped” for comparison. Titanium alloy is assigned to the screws, and the host mandible and graft are assigned with bone material. The effect of the implant's location and material are assessed under the FEA chewing model. The equivalent Von Mises stress distribution on the plate is analyzed before and after bone healing. Additionally, the posterior and anterior gap created between the bone graft and the mandibular bone on the before-healing condition is also assessed.

FIG. 18 shows Von Mises stress distribution after osteotomy (before bone healing) for different implant locations (“straight,” “angled,” and “warped”) and device material choices (Ti64 and NiTi). The Von Mises stress distribution is obtained after applying boundary conditions for unilateral clenching at the first left molar (M1). As shown, the stress value differs depending on the implant's location and material. The plate deformation increases as the implant changes from a straight location to a warped location, consequently, the stress value increases as well. Since the chewing force exerts a lever arm, provoking the separation of the components, the stress concentration on the plate is observed at the interfragmentary interface level, between the bone graft and the host mandible. For the straight and the angled locations, the maximum stress value is observed in the anterior gap, while for the warped location, increased stress values are seen in both anterior and anterior gaps. Even though this behavior is observed for both materials, Ti64 experiences greater stress than NiTi. Quantitative data can be observed in FIG. 18 Error! Reference source not found. It is clear that the stress increases as the extent of plate angulation and wrapping increase for both materials (NiTi and Ti64). Furthermore, the extent of plate deformation increases due to the increase of gap formation between the graft and the host bone. Hence, changing the location of the implant certainly affects the stability of the fixation site and how the graft moves.

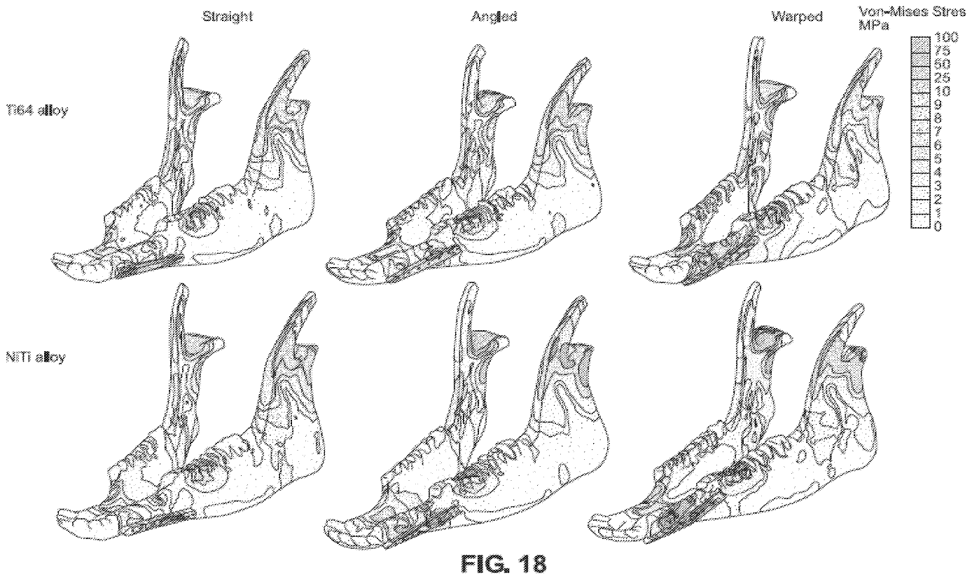

FIG. 19 shows Von Mises stress distribution after bone healing (after bone union) for different implant locations (“straight,” “angled,” and “warped”) and device material choices (Ti64 and NiTi). The evaluation of the plate stress distribution when the bone has healed gives information about the restoration of the normal stress-strain path in the bone. It can be seen that the angulation and bending of the plate reduce the stress in the plate and further stress reduction is observed with the use of NiTi as material.

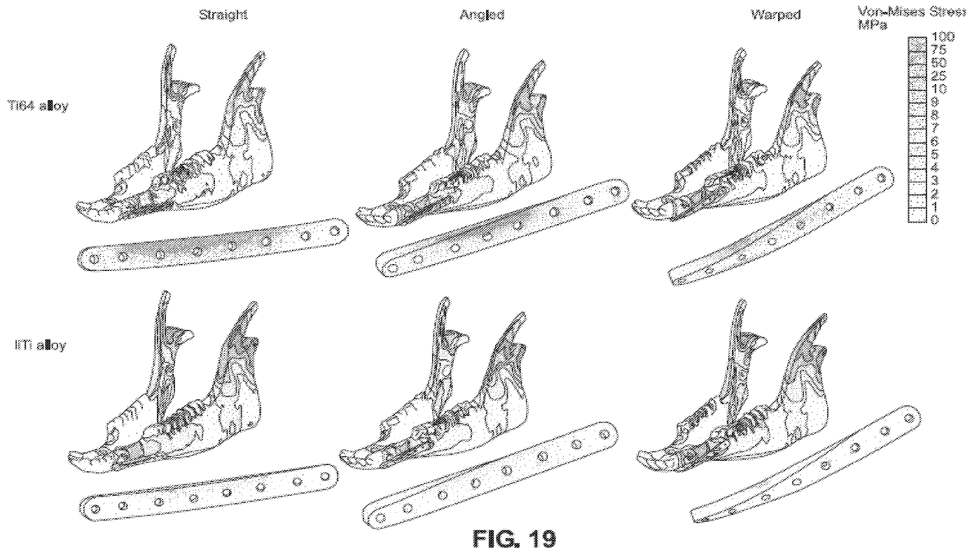

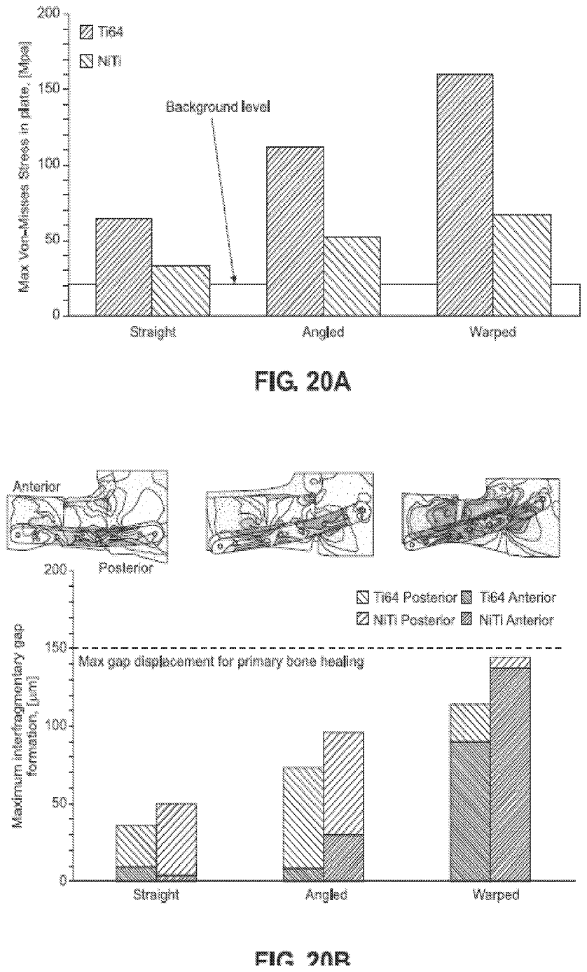

FIGS. 20A and 20B compare the maximum Von Mises stress in the plate and maximum gap formation under before healing condition for the different implant locations (“straight,” “angled,” and “warped”) and device material choices (Ti64 and NiTi) studied. In FIG. 20A, a background level (Von Mises stress around 20 MPa) is shown to provide a reference scale for the after-healing condition. In FIG. 20B, maximum anterior and posterior gap formed during unilateral clenching are compared, augmented deformation of the interfragmentary section is shown above the graph, and the dashed line indicates a reference maximum gap displacement value of about 150 μm for primary bone healing.

It is appreciated that the graft motion changes depending on the plate locations. The increasing angling and warping of the implant promote the formation and increase of the anterior gap superior to the plate, and the posterior gap inferior to the plate. Due to this increasing gap formation, the plate experiences larger deformation and stress concentration at the gap levels. The anterior and posterior observed gaps are smaller than the reported maximum gap for primary bone healing. The straight location plate shows an increased deformation in the upper central region. Likewise, the angled plate shows the same pattern, but in a reduced amount. The angling and bending of the plate reduce the deformation during chewing. Therefore, the warped implant is the most preferred, as it shows significantly reduced stress values (being the least stressed of the three). Considering the same criteria, the more elastic NiTi material is preferred over Ti64.

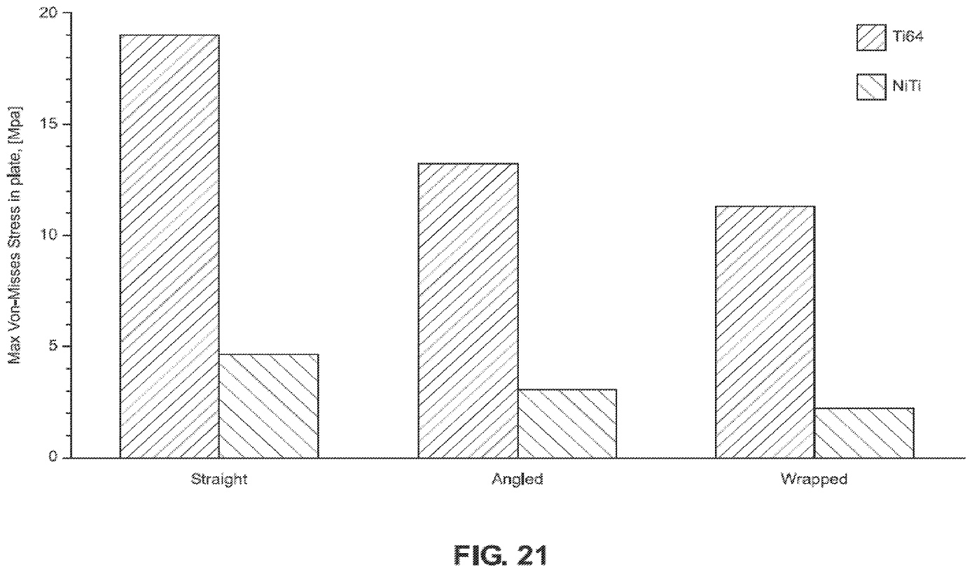

FIG. 21 compares the maximum Von Mises stress in the plate under post bone healing condition for the different implant locations (“straight,” “angled,” and “warped”) and device material choices (Ti64 and NiTi) studied. For reference, the deformation value under the before healing condition is about 20 MPa. The results from the mechanical model under the post healing condition show a reduction of stress values in the fixation plate, as the implant location goes from straight to angled to warped location. This supports that a plate located straight across the diastema opposes the bending and torsion conditions that the mandible experiences under chewing loading, compared to an angled or warped design. This is reflected on the increased stress concentration seen in the straight plate. Hence, by reducing the parallelism of the implant to the diastema, by introducing bending and torsion shapes, the mechanical performance of the implant is enhanced when the plate is no longer needed (post healing).

The results shown in FIGS. 20A, 20B, and 21 help assessing the effects of the implant's location and material selection in the stiffness matching approach. The implant needs to offer mechanical stability and strength while the bone is no longer able to bear the load and to transfer the load to the bone when is healed for stress-shielding reduction. The results show that the fracture fixation stability gets compromised as the implant changes from a straight to an angled to a wrapped position. The increase in the interfragmentary gap shows a change in the interfragmentary section deforms, increasing the motion, which leads to greater stress values in the plate stress. After healing, the reduction of stress concentration, as the implant changes from straight to wrapped, is very promising. Results support that a straight location is not good for handling the bending and torsion conditions that the mandible experience (observed in the increased stress concentration compared to the wrapped location). Reducing the parallelism of the implant (to the diastema), enhances the mechanical performance of the implant, when the plate is no longer needed. Considering the same criteria to minimize/eliminate stress-shielding, these results also show that the more elastic NiTi outperforms Ti64.

These observations demonstrate the applicability of the stiffness-match approach disclosed herein as a new solution to mitigate or eliminate negative effects currently associated with long-term use of skeletal reconstructive devices.

The foregoing description of examples has been presented for purposes of illustration and description. It is not intended to be exhaustive or limiting to the forms described. Numerous modifications are possible in light of the above teachings. Some of those modifications have been discussed, and others will be understood by those skilled in the art. The examples were chosen and described in order to best illustrate principles of various examples as are suited to particular uses contemplated. The scope is, of course, not limited to the examples set forth herein, but can be employed in any number of applications and equivalent devices by those of ordinary skill in the art.

1. A device for bone graft fixation, replacement, or abutment, comprising:
a non-resorbable portion made of a non-resorbable biocompatible material; and
a resorbable portion made of a resorbable biocompatible material joined to the non-resorbable portion, wherein the resorbable portion is resorbed in a pre-determined timing such that mechanical properties of the device are modulated to avoid stress shielding of a bone and to control stress concentrations in the device below a predetermined level to avoid failure of the device.

2. The device of claim 1, wherein the non-resorbable portion and the resorbable portion are joined via press fitting, welding, explosive joining, impact welding comprising vaporizing foil actuator welding, magnetic impulse welding, laser driven impact welding, or a combination thereof, pulsed and continuous wave laser welding, electron beam welding, friction welding, ultrasonic welding, ultrasonic additive manufacturing, cold spray, casting, infusion, jetting, brazing, soldering, diffusion joining, transient liquid bonding, additive manufacturing or a combination thereof.

3. The device of claim 1 is substantially free of intermediate phase formation between the non-resorbable portion and the resorbable portion.

4. The device of claim 1, wherein the non-resorbable portion and the resorbable portion are substantially free from degradation due to the joining process.

5. The device of claim 1, wherein the non-resorbable portion comprises one or more sockets configured to receive the resorbable portion.

6. The device of claim 3, wherein the non-resorbable portion comprises one or more cages configured to at least partially close or contain the resorbable portion inside the one or more sockets.

7. The device of claim 1, wherein the non-resorbable portion comprises one or more porous portions and the resorbable portion is disposed in the one or more porous portions.

8. (canceled)

9. (canceled)

10. The device of claim 1, wherein the non-resorbable biocompatible material is a nickel-titanium based alloy or a shape memory alloy.

11. (canceled)

12. The device of claim 1, wherein the resorbable biocompatible material is selected from one or more of a magnesium-based alloy, a zinc-based alloy, a ceramic-based material.

13. (canceled)

14. (canceled)

15. The device of claim 1, comprising a coating that covers at least a portion of the device to help control the pre-determined timing of the resorption of the resorbable portion.

16. The device of claim 15, wherein the coating is made of a bioactive layer selected from one or more of a calcium phosphate layer, a hydroxyapatite layer, and an osseointegration-enhancing layer.

17. (canceled)

18. The device of claim 15, wherein the coating is functionalized with cells, carriers of drugs, bioactive substances, or a combination thereof.

19. (canceled)

20. (canceled)

21. The device of claim 1, further comprising an interlayer between the non-resorbable portion and the resorbable portion.

22. A method of fabricating a device for bone graft, replacement, or abutment, comprising:
obtaining a non-resorbable portion made of a non-resorbable biocompatible material;
obtaining a resorbable portion made of a resorbable biocompatible material, wherein the resorbable portion is configured to be resorbed in a pre-determined timing such that mechanical properties of the device are modulated to avoid stress shielding of a bone and to control stress concentrations in the device below a predetermined level to avoid failure of the device; and
joining the non-resorbable portion and the resorbable portion to form the device.

23. The method of claim 22, comprising joining the non-resorbable portion and the resorbable portion via press fitting, welding, explosive joining, impact welding comprising vaporizing foil actuator welding, magnetic impulse welding, laser driven impact welding, or a combination thereof, pulsed and continuous wave laser welding, electron beam welding, friction welding, ultrasonic welding, ultrasonic additive manufacturing, cold spray, casting, infusion, jetting, brazing, soldering, diffusion joining, transient liquid bonding, additive manufacturing or a combination thereof.

24. The method of claim 22, comprising coating at least a portion of the device with a coating configured to help control the pre-determined timing of the resorption of the resorbable portion.

25. The method of claim 22, wherein obtaining the non-resorbable portion and the resorbable portion comprises fabricating the non-resorbable portion and the resorbable portion with personalized patient-specific shapes and dimensions.

26. The method of claim 22, wherein joining comprises joining the non-resorbable portion and the resorbable portion such that the device is substantially free of intermediate phase formation between the non-resorbable portion and the resorbable portion.

27. The method of claim 22, wherein joining comprises joining the non-resorbable portion and the resorbable portion such that the non-resorbable portion and the resorbable portion are substantially free from degradation due to the joining process.

28. The method of claim 22, further comprising disposing an interlayer between the non-resorbable portion and the resorbable portion before joining the non-resorbable portion and the resorbable portion.
